(19) United States
(12) Patent Application Publication
(10) Pub. No.: US 2019/0290186 A1
(43) Pub. Date: Sep. 26, 2019

(54) PERSPIRATION STATE ESTIMATION DEVICE, PERSPIRATION STATE ESTIMATION METHOD, AND PERSPIRATION STATE ESTIMATION PROGRAM

(71) Applicant: SHARP KABUSHIKI KAISHA, Sakai City, Osaka (JP)

(72) Inventors: YOSHIHISA ADACHI, Sakai City (JP); YASUHIRO HARADA, Sakai City (JP); HITOSHI NAKAMURA, Sakai City (JP); KAZUYUKI MATSUOKA, Sakai City (JP)

(21) Appl. No.: 16/305,874

(22) PCT Filed: Apr. 18, 2017
(86) PCT No.: PCT/JP2017/015519

§ 371 Date: Nov. 29, 2018

(30) Foreign Application Priority Data
Jun. 3, 2016 (JP) 2016-112254

(51) Int. Cl. A61B 5/00

(52) U.S. Cl. CPC A61B 5/4266; A61B 5/7278; A61B 2562/029; A61B 2560/0252; A61B 5/7275

(57) ABSTRACT

The perspiration state of a site of a living body including at least a part other than a local part of which the perspiration state is measured is accurately estimated. A perspiration data estimation device includes: a comparing section comparing local perspiration data acquired by a perspiration sensor with a first perspiration pattern indicating progression of the amount of perspiration on the local part over time; and a perspiration state estimating section estimating the amount of perspiration on the whole body on the basis of a result of the comparison at the comparing section and a second perspiration pattern indicating progression of the perspiration state of the whole body over time.

### TECHNICAL FIELD

The disclosure described below relates to a perspiration state estimation device and the like.

### BACKGROUND ART

The number of consecutive hot days, an abnormally high temperature day, or the like has recently increased because of effect of a heat island phenomenon, global warming, or the like. This increases heat stress in a general environment and increases the number of heatstroke patients transported by ambulance, which has become an issue of public concern.

A significant key to noninvasively know the risk of heatstroke is perspiration, which is the only way of heat dissipation in a living body. One method of knowing the risk of heatstroke using perspiration is detection of a rate of decrease in body water with respect to the weight of a user.

To acquire a rate of decrease in body water with respect to the weight, the amount of perspiration on the whole body is needed to be known. A sensor detecting the amount of perspiration is preferably as small as possible in consideration of comfortability when attached to the user. In this case, the amount of perspiration on the whole body is estimated on the basis of the amount of local perspiration measured at one part of the body.

PTL 1 discloses a perspiration amount measurement patch that measures the amount of perspiration on the body of a user (subject) per unit area to know the amount of perspiration on the whole body. This patch is applied to a part to be measured of the subject's body and measures the amount of perspiration on the part to be measured. Then, the measured amount of perspiration is multiplied by a prescribed coefficient to acquire the amount of perspiration on the whole body (whole body perspiration amount).

### CITATION LIST

### Patent Literature

PTL 1: JP 2010-046196 A (published on Mar. 4, 2010)

### SUMMARY

### Technical Problem

In PTL 1, it is described concerning the aforementioned prescribed coefficient that accurate calculation is difficult because of variations in the surface area of the skin, the weight, and other factors. In PTL 1, a method of more accurately measuring the amount of perspiration is described. The method calculates an appropriate coefficient by measuring, by the user of the patch, a decrement from the weight before playing sports, for example, and acquiring a ratio between the amount of perspiration on the part to be measured and the decrement. In PTL 1, it is described that factors of the user for acquiring the coefficient include sex, age, weight, and height.

Unfortunately, the timing of starting perspiration and the amount of perspiration differ depending on the part of the body. Thus, even for the same user, the appropriate value of the prescribed coefficient may vary with the time period elapsed from when the user is in an environment causing perspiration. In addition, the environment around the user, the body-build of the user, or the like may vary the relationship between the amount of local perspiration and the amount of perspiration on the whole body. Thus, the perspiration amount measurement patch disclosed in PTL 1 may be difficult to accurately estimate the whole body perspiration amount.

In the light of the foregoing problem, an object of the disclosure described below is to achieve a perspiration state estimation device capable of accurately estimating a perspiration state of a site of a living body including at least a part other than a local part of which the perspiration state is measured.

### Solution to Problem

To solve the above problem, a perspiration state estimation device according to an aspect of the disclosure is capable of being connected to a local perspiration data acquiring unit and an environment data acquiring unit in a communicable manner, the local perspiration data acquiring unit being configured to acquire local perspiration data indicating a perspiration state of a local part of a living body, the environment data acquiring unit being configured to acquire environment data indicating a state of an environment where the living body is present, and includes: a comparing section configured to compare (1) the local perspiration data acquired by the local perspiration data acquiring unit with (2) a first perspiration pattern correlated with at least either of attribute data indicating an attribute of the living body and the environment data acquired by the environment data acquiring unit, the first perspiration pattern indicating progression of the perspiration state of the local part over time; and an estimating section configured to estimate a perspiration state of a site of the living body on a basis of a result of the comparison at the comparing section and a progression relating pattern indicating a second perspiration pattern indicating progression of the perspiration state of the site over time or a progression relating pattern indicating a relationship between the first perspiration pattern and the second perspiration pattern, the site including at least a part other than the local part.

A perspiration state estimation method according to an aspect of the disclosure includes: a local perspiration data acquiring step of acquiring local perspiration data indicating a perspiration state of a local part of a living body; an environment data acquiring step of acquiring environment data indicating a state of an environment where the living body is present; a comparing step of comparing (1) the local perspiration data acquired in the local perspiration data acquiring step with (2) a first perspiration pattern correlated with at least either of attribute data indicating an attribute of the living body and the environment data acquired in the environment data acquiring step, the first perspiration pattern indicating progression of the perspiration state of the local part over time; and an estimating step of estimating a perspiration state of a site of the living body on a basis of a result of the comparison in the comparing step and a progression relating pattern indicating a second perspiration pattern indicating progression of the perspiration state of the site over time or a progression relating pattern indicating a relationship between the first perspiration pattern and the second perspiration pattern, the site including at least a part other than the local part.

### Advantageous Effects of Disclosure

The perspiration state estimation device or the perspiration state estimation method according to an aspect of the disclosure exhibits the advantageous effect of accurately estimating a perspiration state of a site of a living body including at least a part other than a local part of which the perspiration state is measured.

### BRIEF DESCRIPTION OF DRAWINGS

FIG. 1 is a diagram illustrating an example of a configuration of a user support system according to a first embodiment.

FIG. 2A is a diagram illustrating an example of perspiration patterns stored in a storage. FIG. 2B is a diagram illustrating a ratio of a first perspiration pattern to a second perspiration pattern illustrated in FIG. 2A. FIG. 2C is a diagram for describing estimation of a perspiration state in a perspiration state estimation device.

FIG. 3 is a flowchart of an example of a perspiration state estimation method according to the first embodiment.

FIG. 4 is a diagram illustrating an example of a configuration of a user support system according to a second embodiment.

FIG. 5 is a diagram for describing estimation of a perspiration state in a perspiration state estimation device according to the second embodiment.

FIG. 6 is a diagram illustrating an example of perspiration patterns identified by a perspiration pattern identifying section according to a modification of the second embodiment.

FIG. 7 is a flowchart of an example of a perspiration state prediction method according to the modification of the second embodiment.

FIG. 8 is a diagram illustrating an example of a configuration of a user support system according to a third embodiment.

FIG. 9 is a diagram illustrating an example of a configuration of a user support system according to a fourth embodiment.

FIG. 10A is a graph showing a first perspiration pattern and a second perspiration pattern in the case of a temperature of 20° C. FIG. 10B is a graph showing progression over time of a ratio between the first perspiration pattern and the second perspiration pattern in the case of the temperature of 20° C. FIG. 10C is a graph showing a first perspiration pattern and a second perspiration pattern in the case of a temperature of 25° C. FIG. 10D is a graph showing progression over time of a ratio between the first perspiration pattern and the second perspiration pattern in the case of the temperature of 25° C. FIG. 10E is a graph showing a first perspiration pattern and a second perspiration pattern generated by a pattern generating section in the case of a temperature of 23° C. FIG. 10F is a graph showing progression over time of a ratio between the first perspiration pattern and the second perspiration pattern in the case of the temperature of 23° C.

FIG. 11 is a flowchart of an example of a perspiration state prediction method according to the fourth embodiment.

FIG. 12 is a diagram illustrating an example of a configuration of a user support system according to a fifth embodiment.

FIG. 13A is a diagram illustrating an example of perspiration patterns in the case of a prescribed MET value. FIG. 13B is a diagram illustrating a ratio between a first perspiration pattern and a second perspiration pattern on the basis of the perspiration patterns illustrated in FIG. 13A.

FIG. 14 is a flowchart of an example of a perspiration state prediction method according to the fifth embodiment.

FIG. 15 is a diagram illustrating an example of a configuration of a user support system according to a sixth embodiment.

FIG. 16 is a diagram for describing estimation of a perspiration state in a perspiration state estimation device according to the sixth embodiment.

FIG. 17 is a flowchart of an example of a perspiration state estimation method according to the sixth embodiment.

### DESCRIPTION OF EMBODIMENTS

### First Embodiment

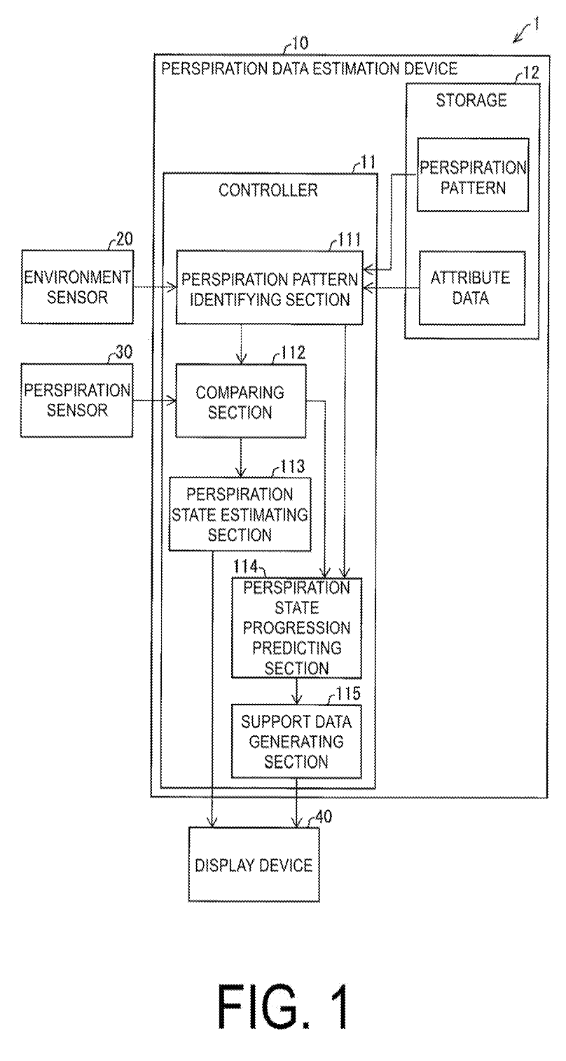

An embodiment of the disclosure will be described below with reference to FIG. 1 to FIG. 3.

### User Support System

FIG. 1 is a diagram illustrating an example of a configuration of a user support system 1 according to the present embodiment. The user support system 1 estimates the amount of perspiration as a perspiration state of a user (living body) and supports management of the physical condition of the user on the basis of a result of the estimation. As illustrated in FIG. 1, the user support system 1 includes a perspiration data estimation device 10 (perspiration state estimation device), an environment sensor 20 (environment data acquiring unit), a perspiration sensor 30 (local perspiration data acquiring unit), and a display device 40. The perspiration data estimation device 10 is connected to the environment sensor 20, the perspiration sensor 30, and the display device 40 in a communicable manner. Note that the perspiration data estimation device 10 will be described later.

The environment sensor 20 acquires data indicating at least either of temperature and humidity in an environment where the user is present as environment data, and transmits the data to the perspiration data estimation device 10. Examples of the environment sensor 20 of the present embodiment include a temperature sensor and a humidity sensor. The environment sensor 20 may be an ultra violet (UV) sensor measuring the amount of ultraviolet rays radiated to the user or an illumination sensor measuring the amount of illumination radiated to the user. The following description is provided, assuming that the environment sensor 20 is a temperature sensor.

Note that the perspiration data estimation device 10 may be connected to a receiving device (not illustrated) (environment data acquiring unit) capable of acquiring environment data, instead of the environment sensor 20. In this case, the receiving device retrieves environment data from an external device storing environment data. The environment data may be, for example, weather information in an environment (area) where the user is present. The receiving device retrieves environment data from the external device via a network line.

The perspiration sensor 30 acquires local perspiration data indicating the amount of perspiration on a local part of the user. In the present embodiment, the description is provided, assuming that the perspiration sensor 30 is a perspiration amount sensor acquiring the amount of perspiration on the left forearm of the user, that is, the “local part” being a part of which the local perspiration data is to be acquired is the left forearm of the user's body. Note that the “left forearm” refers to a part from the wrist to the elbow of the left arm.

The display device 40 displays perspiration state data generated by the perspiration data estimation device 10 and indicating the amount of perspiration on the whole body and support data indicating measures to reduce the possibility that the user gets into poor physical condition. The user support system 1 may include any presentation device as long as the device can present, to the user, content of the perspiration state data and the support data, and may include, for example, a speaker outputting the content in voice form as the presentation device, instead of the display device 40.

### Perspiration State Estimation Device

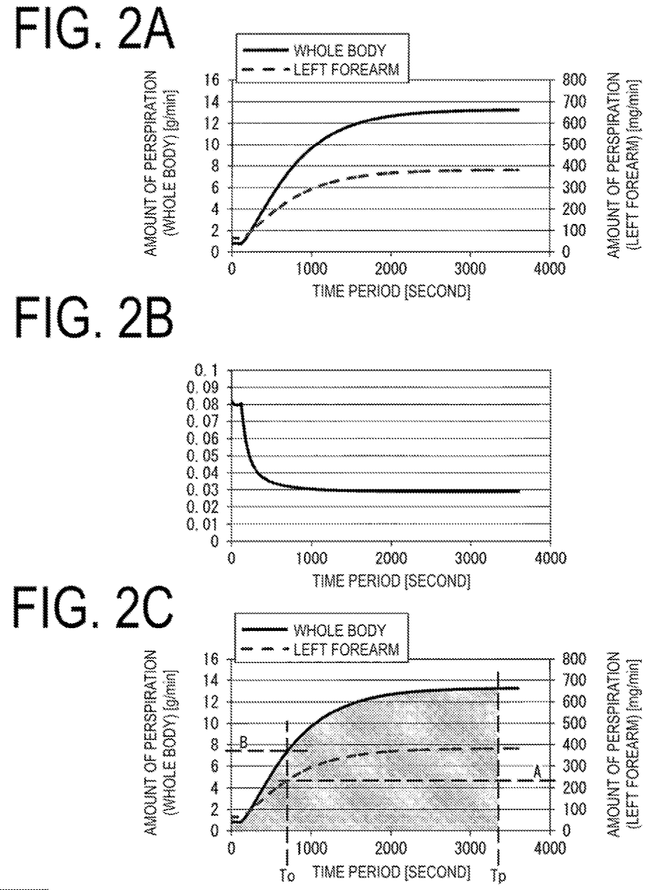

Next, the perspiration data estimation device 10 will be described with reference to FIG. 1, and FIGS. 2A, 2B, and 2C. The perspiration data estimation device 10 estimates the amount of perspiration on the user's whole body, and includes a controller 11 and a storage 12 as illustrated in FIG. 1. The perspiration data estimation device 10 can be connected to the perspiration sensor 30 and the environment sensor 20 as illustrated in FIG. 1.

The controller 11 controls the entire perspiration data estimation device 10, and includes a perspiration pattern identifying section 111 (identifying section), a comparing section 112, a perspiration state estimating section 113 (estimating section), a perspiration state progression predicting section 114, and a support data generating section 115. A specific configuration of the controller 11 will be described later.

The storage 12 stores various control programs and the like executed by the controller 11, and is constituted by a nonvolatile storage device, such as a hard disk and a flash memory. The storage 12 stores, for example, perspiration patterns being a target of identification at the perspiration pattern identifying section 111 and attribute data to be looked up at the time of the identification. The attribute data indicates user's attributes including at least any of the body-build, age, sex, and cloth information of the user. The body-build of the user is an attribute relating to the body condition of the user, such as height, weight, and body fat percentage. The cloth information is an attribute relating to the cloth worn by the user, such as a long-sleeved cloth and a short-sleeved cloth. The perspiration patterns will be described later.

Note that the perspiration patterns and the attribute data are not necessarily stored in the storage 12 in advance and may be present when the perspiration pattern identifying section 111 performs perspiration pattern identification processing. In this case, the perspiration patterns and the attribute data may be input from an input section (not illustrated) receiving input from the user at the time of the identification processing, for example.

### Configuration of Controller

The perspiration pattern identifying section 111 identifies a first perspiration pattern used for comparison with local perspiration data at the comparing section 112 and a second perspiration pattern (progression relating pattern) used for estimation of the amount of perspiration on the whole body at the perspiration state estimating section 113. The first perspiration pattern of the present embodiment indicates progression of the amount of perspiration on the user's left forearm over time. The second perspiration pattern indicates progression of the amount of perspiration on the user's whole body over time. In the following description, the first perspiration pattern and the second perspiration pattern are simply referred to as a perspiration pattern when necessary.

Note that the first perspiration pattern is not limited to this example and may indicate progression of the amount of perspiration on any local part of the user's body over time. In other words, the first perspiration pattern may indicate progression of the amount of perspiration on any part other than the left forearm, such as the right forearm, left ankle, right ankle, left thigh, and right thigh, over time. The second perspiration pattern may indicate progression of the amount of perspiration on a site of the user's body including at least a part other than the local part (a site, different from the local part, of the user's body) over time. In other words, in a case where the first perspiration pattern is for the left forearm, the second perspiration pattern may indicate progression of the amount of perspiration on the whole body or on any of the parts other than the left forearm or a plurality of parts among the parts, over time.

In specific, the perspiration pattern identifying section 111 identifies at least either of (1) a first perspiration pattern corresponding to the user's attribute data among a plurality of first perspiration patterns correlated with a plurality of predetermined attribute values indicating an attribute and (2) a first perspiration pattern corresponding to the environment data acquired by the environment sensor 20 among a plurality of first perspiration patterns correlated with a plurality of predetermined environment values indicating a prescribed environment state.

In other words, (1) in a case where perspiration patterns correlated only with the attribute data are prepared, the perspiration pattern identifying section 111 uses only attribute data to identify a perspiration pattern corresponding to the attribute data. (2) In a case where perspiration patterns correlated only with the environment data are prepared, the perspiration pattern identifying section 111 uses only environment data to identify a perspiration pattern corresponding to the environment data. (3) In a case where perspiration patterns correlated with both the attribute data and the environment data are prepared, the perspiration pattern identifying section 111 uses both attribute data and environment data to identify a perspiration pattern corresponding to the attribute data and the environment data. Note that the present embodiment is described, assuming the case (3) above.

An example of a perspiration pattern identified by the perspiration pattern identifying section 111 will be described with reference to FIGS. 2A, 2B, and 2C. FIG. 2A illustrates of an example perspiration patterns stored in the storage 12. The first perspiration pattern is indicated by the broken line in FIG. 2A. The second perspiration pattern is indicated by the solid line in FIG. 2A. The first and second perspiration patterns illustrated in FIG. 2A are a group of perspiration patterns that are correlated with attribute values and/or environment values and that are a target of identification at the perspiration pattern identifying section 111.

FIG. 2B illustrates a ratio of the first perspiration pattern to the second perspiration pattern illustrated in FIG. 2A. As illustrated in FIG. 2B, the ratio indicates progression over time and varies with the time period (referred to as time for convenience) elapsed from the start of measurement. This is because the timing of starting perspiration and the amount of perspiration after the user is in an environment causing perspiration differ depending on the part of the body.

The storage 12 stores the perspiration patterns correlated with the predetermined environment values. For example, perspiration patterns for temperatures of 20° C., 30° C., and 40° C. are prepared. A plurality of perspiration patterns for temperatures other than these temperatures may of course be prepared. Regarding an unprepared temperature, the perspiration pattern identifying section 111 may generate a perspiration pattern through interpolation processing (interpolation or extrapolation) using the prepared perspiration patterns. Alternatively, as in an embodiment described later, the perspiration pattern may be expanded, factoring in activity data of the user.

The storage 12 also stores the perspiration patterns correlated with the predetermined attribute values indicating the attribute of the user. For example, for the attribute “age”, a perspiration pattern correlated with each of a plurality of attribute values (such as teens, 20 s, . . . ) may be prepared. For the attribute “sex”, a perspiration pattern correlated with each of attribute values “male” and “female” may be prepared. For the attribute “body fat percentage”, a perspiration pattern correlated with each of a plurality of attribute values (such as a body fat percentage of 10%, 20%, . . . ) may be prepared. Perspiration patterns correlated with yet another attribute may be prepared. Note that, similar to the perspiration patterns correlated with environment values, the attribute values of the age, body fat percentage, or the like can be expanded through the aforementioned interpolation processing or using the activity data.

Note that the perspiration pattern is not required to be correlated with attribute values indicating a plurality of attributes and may be correlated with an attribute value indicating only one attribute (for example, age).

The perspiration pattern identifying section 111 identifies a perspiration pattern corresponding to a temperature (for example, 25° C.) indicated by the environment data acquired by the environment sensor 20 and values (age: 45, sex: male, body fat percentage: 20%) indicated by the attribute data stored in the storage 12, for example.

Note that in the present embodiment, with the perspiration patterns prepared in the storage 12, the perspiration pattern identifying section 111 uses the attribute data and the environment data to identify a perspiration pattern among the perspiration patterns; however, no perspiration pattern may be prepared. In this case, a mathematical expression for calculating a perspiration pattern is prepared in the storage 12. The perspiration pattern identifying section 111 may insert a value indicated by the attribute data and/or the environment data into the mathematical expression to identify a perspiration pattern used by the comparing section 112 and the perspiration state estimating section 113.

The comparing section 112 compares the local perspiration data acquired by the perspiration sensor 30 with the first perspiration pattern identified by the perspiration pattern identifying section 111. In the present embodiment, the first perspiration pattern used for the comparison is correlated with both the attribute data and the environment data. As described above, the first perspiration pattern may be correlated only with the attribute data or only with the environment data in some cases.

FIG. 2C is a diagram for describing estimation of the amount of perspiration on the whole body in the perspiration data estimation device 10. The comparing section 112 acquires the local perspiration data acquired by the perspiration sensor 30 from the perspiration sensor 30 and identifies time To, corresponding to the value indicated by the local perspiration data (value A in FIG. 2C), in the identified first perspiration pattern. The horizontal axis of the graph showing the first and second perspiration patterns indicates a time period elapsed from the start of measuring the amount of perspiration indicated by the first and second perspiration patterns. Thus, the time To is one point in the time period elapsed from the start of the measurement.

The perspiration state estimating section 113 estimates the amount of perspiration on the user's whole body on the basis of the second perspiration pattern and the time identified through the comparison at the comparing section 112 and corresponding to the value indicated by the local perspiration data in the first perspiration pattern. In specific, in FIG. 2C, the amount B of perspiration corresponding to the time To, acquired as a result of the comparison, in the second perspiration pattern is estimated as the amount of perspiration on the whole body.

Note that the storage 12 may store a progression relating pattern corresponding to the first perspiration pattern and indicating a relationship between the first perspiration pattern and the second perspiration pattern as a progression relating pattern relating to progression of the amount of perspiration on the user's whole body over time, instead of the second perspiration pattern. An example of such a progression relating pattern is a pattern indicating progression of a ratio between the first perspiration pattern and the second perspiration pattern over time (for example, the pattern illustrated in FIG. 2B). This pattern indicates progression of a ratio between the first perspiration pattern and the second perspiration pattern correlated with the same attribute data and/or environment data as the attribute data and/or environment data correlated with the first perspiration pattern, over time. In this case, the perspiration state estimating section 113 multiplies the local perspiration data by the ratio at the time To to estimate the amount of perspiration on the whole body.

The perspiration state estimating section 113 causes the display device 40 to display the estimated amount of perspiration on the whole body at the time To, indicated by the perspiration state data, for example. Note that the perspiration state estimating section 113 may calculate a cumulative value that will be described below (herein, a cumulative value of the amounts of perspiration on the whole body until the time To) and cause the display device 40 to display the calculated cumulative value.

The perspiration state progression predicting section 114 predicts progression of the amount of perspiration on the whole body over time after the acquisition of the local perspiration data at the perspiration sensor 30, on the basis of the comparison result from the comparing section 112 and the second perspiration pattern. In other words, the perspiration state progression predicting section 114 predicts progression of the amount of perspiration on the whole body over time after the time To illustrated in FIG. 2A (i.e., for a time to come after the time To).

For example, the perspiration state progression predicting section 114 predicts, from the perspiration pattern identified by the perspiration pattern identifying section 111, (1) in how many minutes from the time To and how much the amount of perspiration will become, (2) how many minutes it will take for the amount of perspiration to reach a prescribed amount of perspiration (prescribed value) (when the amount of perspiration will reach a prescribed amount of perspiration), and the like.

The amount of perspiration to be compared with the prescribed amount of perspiration may be the amount of perspiration per unit time period (for example, the amount of perspiration per minute) indicated by the second perspiration pattern, or may be a cumulative value of the amounts of perspiration after time 0 (i.e., the start of the measurement) indicated in the second perspiration pattern. This cumulative value is calculated as the area surrounded by the time period axis (horizontal axis; y=0), prescribed time Tp (x=Tp) on the horizontal axis of the graph showing the perspiration patterns, and the second perspiration pattern (the gray portion of FIG. 2C).

In general, in a case where water in a body decreases by a prescribed amount, the physical condition changes for the worse. In specific, in a case where the amount of water lost from the body is less than 2% of the weight, the user only feels thirsty. In a case where the amount is 2% or greater, especially approximately from 3 to 4%, the user may feel something unusual, such as lack of appetite and fatigue. In a case where the amount of water lost is 5% or greater of the weight, serious abnormality, such as speech disturbance and convulsions, may occur.

In a case where the user's weight is acquired as an attribute, the perspiration data estimation device 10 determines the amount of water equal to, for example, 2% of the user's weight as a threshold. In this case, the perspiration state progression predicting section 114 calculates the cumulative value of the amounts of perspiration at times on the horizontal axis after the time 0 (the above-described area) in the identified second perspiration pattern. Then, the time Tp when the cumulative value is equal to or greater than the threshold is identified. That is, the perspiration state progression predicting section 114 can predict that in a case where the user remains in the current environment, the physical condition may change for the worse in Tp−To minutes.

The support data generating section 115 generates support data on the basis of the progression of the amount of perspiration on the whole body over time predicted by the perspiration state progression predicting section 114, and causes the display device 40 to display the data. The support data generated by the support data generating section 115 contains notification of time when the possibility of heatstroke increases, time when the user should drink water, or the like.

In a case where the perspiration state progression predicting section 114 predicts that the physical condition of the user may change for the worse in Tp−To minutes, for example, the support data generating section 115 generates support data indicating the content “Possibility of heatstroke in Tp−To minutes. Please hydrate within the time limit.”.

### Perspiration State Estimation Method

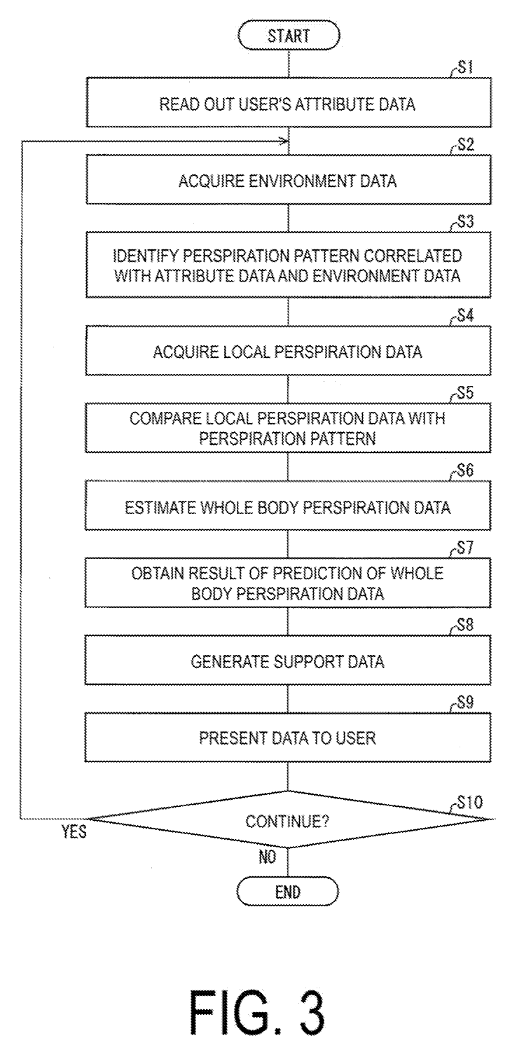

Next, a method of estimating the amount of perspiration on the whole body will be described with reference to FIG. 3. FIG. 3 is a flowchart of an example perspiration amount estimation method (control method for the perspiration data estimation device 10 and the like) according to the present embodiment.

As illustrated in FIG. 3, the perspiration pattern identifying section 111 reads out attribute data of the user from the storage 12 (S1). Next, the environment sensor 20 acquires environment data, and the perspiration pattern identifying section 111 acquires the environment data from the environment sensor 20 (S2; environment data acquiring step). The environment sensor 20 may acquire environment data and transmit the data to the perspiration pattern identifying section 111 in response to a request from the perspiration pattern identifying section 111 or may transmit environment data nearest to the time of the request among accumulated environment data to the perspiration pattern identifying section 111, for example.

The perspiration pattern identifying section 111 identifies a perspiration pattern correlated with the read out attribute data and the environment data acquired from the environment sensor 20 among a plurality of perspiration patterns stored in the storage 12 (S3). The perspiration pattern identified by the perspiration pattern identifying section 111 is used as the first perspiration pattern by the comparing section 112 or as the second perspiration pattern by the perspiration state estimating section 113.

Next, the perspiration sensor 30 acquires local perspiration data (S4; local perspiration data acquiring step). Then, the comparing section 112 acquires the local perspiration data from the perspiration sensor 30. Similar to the environment sensor 20, the perspiration sensor 30 may acquire local perspiration data and transmit the data to the comparing section 112 in response to a request from the comparing section 112 or may transmit local perspiration data nearest to the time of the request among accumulated local perspiration data to the comparing section 112, for example. Then, the comparing section 112 compares the acquired local perspiration data with the identified first perspiration pattern and transmits a result of the comparison (for example, the time To illustrated in FIG. 2C) to the perspiration state estimating section 113 (S5; comparing step). Then, the perspiration state estimating section 113 estimates data indicating the amount of perspiration on the user's whole body (whole body perspiration data) on the basis of the comparison result and the second perspiration pattern (S6; estimating step).

The perspiration state progression predicting section 114 predicts progression of the amount of perspiration on the whole body over time after the acquisition of the local perspiration data on the basis of the comparison result and the second perspiration pattern, and transmits a result of the prediction to the support data generating section 115 (S7). The support data generating section 115 generates support data on the basis of the prediction result (S8) and causes the display device 40 to display the data (S9). At this time, the perspiration state estimating section 113 causes the display device 40 to display the estimated perspiration state data. Thereafter, on the basis of a command from the user, for example, the controller 11 goes back to the step S2 if the steps S2 to S9 are performed again (YES in S10), or ends the procedure if those steps are not performed again (NO in S10)

Note that (1) the steps S2 and S3 and (2) the step S4 may be performed simultaneously, or the steps (1) may be performed after the step (2). In addition, (3) the step S6 and (4) the steps S7 and S8 may be performed simultaneously, or the step (3) may be performed after the steps (4).

### Main Advantageous Effect

The perspiration data estimation device 10 compares the local perspiration data acquired by the perspiration sensor 30 with the first perspiration pattern identified by the perspiration pattern identifying section 111 to identify the time To in the first perspiration pattern. In addition, the amount of perspiration on the whole body at the time To is estimated on the basis of the identified time To and the second perspiration pattern. Thus, the amount of perspiration on the user's whole body can be accurately estimated from the amount of perspiration on the user's local part being a target of the acquisition at the perspiration sensor 30. The perspiration data estimation device 10 can also predict the amount of perspiration on the whole body after the time To when the local perspiration data is acquired.

Furthermore, in the perspiration data estimation device 10, the perspiration pattern identifying section 111 identifies the perspiration pattern correlated with the attribute data indicating the current attribute of the user and/or the environment data indicating the state of the environment where the user is present. Thus, the perspiration pattern identifying section 111 can identify the perspiration pattern appropriate for an individual difference of the user and/or the environment where the user is present. Accordingly, the perspiration data estimation device 10 can estimate the amount of perspiration on the whole body in consideration of the individual difference and/or the environment.

Furthermore, the perspiration data estimation device 10 generates the support data on the basis of the amount of perspiration on the whole body and presents the data to the user. That is, before a health problem, such as a change of the physical condition to a worse condition, occurs, the perspiration data estimation device 10 can present, to the user, the time when the problem is highly likely to occur. Thus, the user can take measures to prevent such a change of the physical condition at an appropriate time.

### Second Embodiment

A second embodiment of the disclosure will be described below with reference to FIG. 3 to FIG. 5. Note that, for convenience of explanation, components illustrated in respective embodiments are designated by the same reference numerals as those having the same function, and the descriptions of these components will be omitted.

### Configuration of Perspiration State Estimation Device

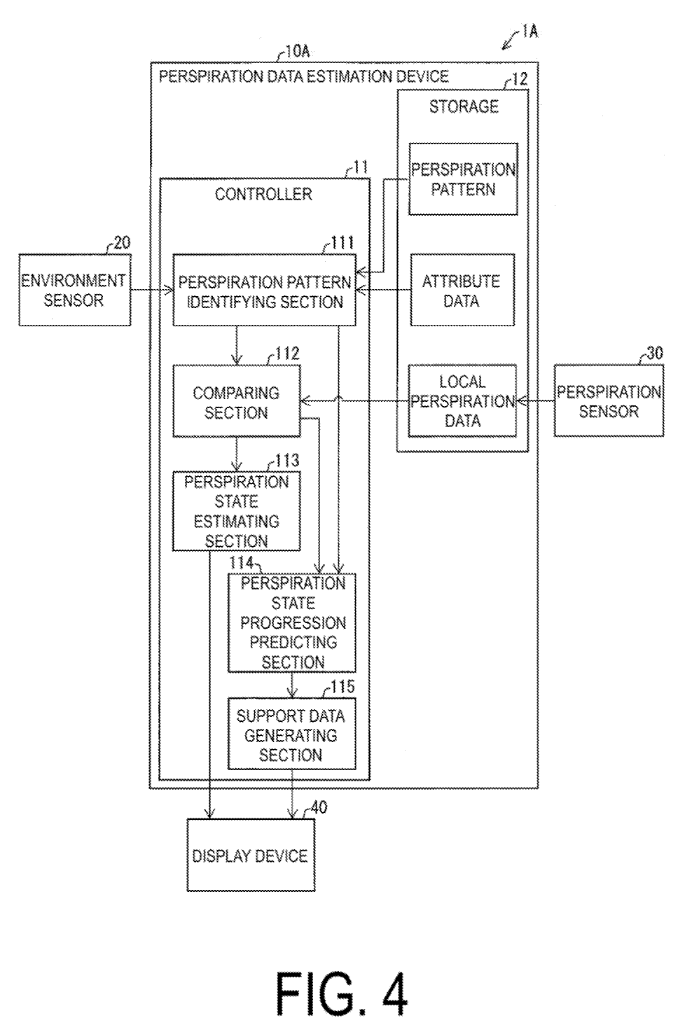

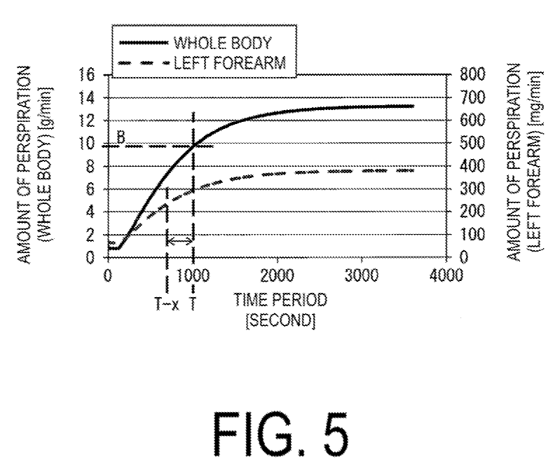

First, an example of a perspiration data estimation device 10A (perspiration state estimation device) according to the present embodiment will be described with reference to FIG. 4. FIG. 4 is a diagram illustrating an example of a configuration of a user support system 1A according to the present embodiment. The user support system 1A includes the perspiration data estimation device 10A, which differs from the user support system 1 of the first embodiment. FIG. 5 is a diagram for describing estimation of the amount of perspiration on the whole body in the perspiration data estimation device 10A.

In specific, in the perspiration data estimation device 10 of the first embodiment, the comparing section 112 acquires the local perspiration data acquired by the perspiration sensor 30 and compares the local perspiration data with the first perspiration pattern identified by the perspiration pattern identifying section 111. On the other hand, in the perspiration data estimation device 10A of the present embodiment, the perspiration sensor 30 acquires local perspiration data at a plurality of times, and the comparing section 112 compares the plural pieces of local perspiration data acquired by the perspiration sensor 30 with the first perspiration pattern.

More specifically, in the perspiration data estimation device 10A, the perspiration sensor 30 temporarily stores, in the storage 12, the plural pieces of local perspiration data acquired at the plural times (in the example in FIG. 5, a plurality of times between time T and time corresponding to time T−x before the time T on the horizontal axis of the graph showing perspiration patterns, inclusive). The comparing section 112 obtains a fitted curve (characteristics over time acquired from the plural pieces of local perspiration data) by, for example, the least squares method for the plural pieces of local perspiration data acquired at the plural times by the perspiration sensor 30. Then, the comparing section 112 compares (fits) the obtained fitted curve with (to) the first perspiration pattern identified by the perspiration pattern identifying section 111. Note that in a case where there are two pieces of local perspiration data as in the example in FIG. 5, a straight line connecting those two pieces of data may be used instead of a fitted curve.

The comparing section 112 considers, as time To, time having the best fit on the horizontal axis (time having the highest level of coincidence on the horizontal axis, that is, time at the intersection of the first perspiration pattern and the fitted curve on the horizontal axis) in the fitted curve fitted to the first perspiration pattern. In the example in FIG. 5, assuming that the time T has the highest level of coincidence, the time T is considered as the time To. In this case, the perspiration state estimating section 113 estimates the amount B of perspiration in the second perspiration pattern corresponding to the time To (in the example in FIG. 5, the time T) identified by the comparing section 112, as the amount of perspiration on the whole body. The method of identifying the time To is not limited to this example. For example, time indicating the largest or smallest value on the horizontal axis in the fitted curve after the fitting may be considered as the time To.

Note that the comparing section 112 is not necessarily required to obtain a fitted curve for the plural pieces of local perspiration data and to perform the comparison using the fitted curve, and, for example, may calculate an average value of the amounts of perspiration indicated by the plural pieces of local perspiration data and use the average value in the comparison.

### Living Body State Prediction Method

Next, a method of predicting the amount of perspiration on the whole body in the perspiration data estimation device 10A will be described with reference to FIG. 3. The steps 51 to S3, the step S6, and subsequent steps in FIG. 3 are similar to those in the first embodiment, and descriptions thereof will be omitted.

In S4 in FIG. 3, in the perspiration data estimation device 10A, the perspiration sensor 30 acquires local perspiration data at a plurality of times. The perspiration data estimation device 10A stores the plural pieces of local perspiration data in the storage 12. The comparing section 112 acquires the plural pieces of local perspiration data stored in the storage 12 and obtains, for example, a fitted curve. Then, in S5, the comparing section 112 fits the obtained fitted curve to the first perspiration pattern identified by the perspiration pattern identifying section 111, and identifies the time To being time when the local perspiration data is acquired in the first perspiration pattern (i.e., time corresponding to the local perspiration data in the first perspiration pattern). Thereafter, the amount of perspiration on the whole body is estimated, the amount of perspiration on the whole body over time is predicted, and support data is generated.

### Main Advantageous Effect

The value indicated by the local perspiration data acquired by the perspiration sensor 30 may have a measurement error due to, for example, variations in manufacturing the perspiration sensor 30. In a case where the comparison is performed using the value indicated by one piece of local perspiration data with a measurement error occurring, the measurement error may affect the identification of the time To. Especially in a time period in which the amount of perspiration varies slightly over time, the measurement error may have significant effect.

The perspiration data estimation device 10A uses local perspiration data at a plurality of times for the comparison, so that even if the above-described measurement error occurs, effect of the measurement error that may be exerted on the identification of the time To can be reduced. Thus, even if there are variations in the acquired local perspiration data, the time To can be identified more correctly. Accordingly, the accuracy in estimating the amount of perspiration on the whole body can be improved.

### Modification

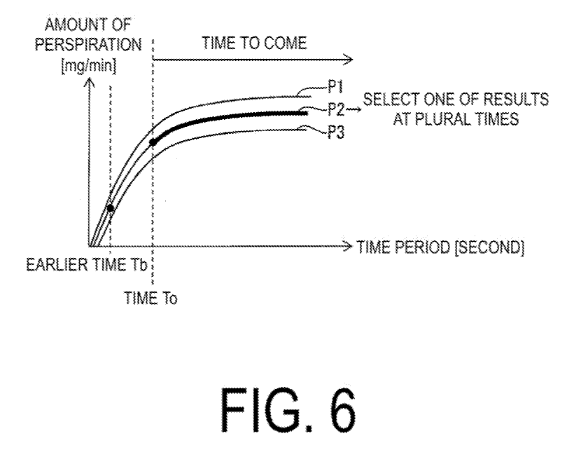

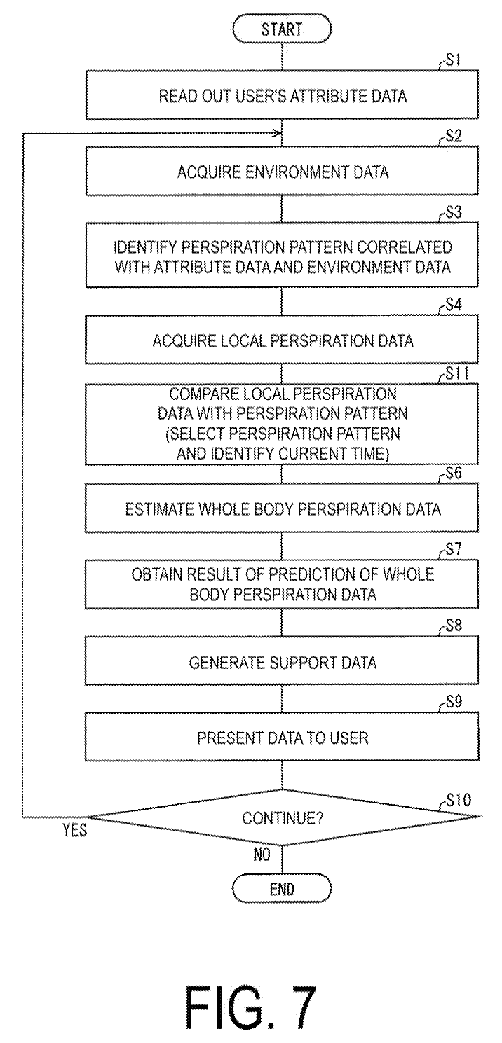

Next, a modification of the second embodiment will be described with reference to FIGS. 4, 6, and 7. FIG. 6 is a diagram illustrating an example of perspiration patterns identified by a perspiration pattern identifying section 111 according to the modification of the second embodiment. FIG. 7 is a flowchart of an example of a method of predicting the amount of perspiration on the whole body according to the modification of the second embodiment.

### Configuration of Perspiration State Estimation Device

In the present modification, the comparison is also performed using plural pieces of local perspiration data acquired by the perspiration sensor 30 at a plurality of times; however, the present modification performs the following processing, which differs from the above-described perspiration data estimation device 10A of the second embodiment. That is, the comparing section 112 uses the plural pieces of local perspiration data acquired by the perspiration sensor 30 to select one perspiration pattern among a plurality of identified first perspiration patterns. The perspiration state estimating section 113 uses a second perspiration pattern corresponding to the first perspiration pattern selected by the comparing section 112 to estimate the amount of perspiration on the whole body. The first perspiration pattern and the second perspiration pattern corresponding to the first perspiration pattern indicate a group of perspiration patterns correlated with the attribute values and/or the environment values. The perspiration state progression predicting section 114 uses the second perspiration pattern corresponding to the first perspiration pattern selected by the comparing section 112 to predict progression of the amount of perspiration on the whole body over time after the acquisition of the local perspiration data.

In specific, the perspiration pattern identifying section 111 identifies a plurality of first and second perspiration patterns among a plurality of perspiration patterns stored in the storage 12, as perspiration patterns correlated with the value indicated by the acquired attribute data and the value indicated by the acquired environment data.

FIG. 6 is a diagram illustrating an example of first perspiration patterns identified by the perspiration pattern identifying section 111 of the present modification. In the example in FIG. 6, three first perspiration patterns P1, P2, and P3 are identified. Second perspiration patterns corresponding to the respective first perspiration patterns P1, P2, and P3 are also identified. The perspiration pattern identifying section 111 identifies the first perspiration patterns in the following manner, for example.

Similar to the first embodiment, the perspiration pattern identifying section 111 identifies one first perspiration pattern correlated with the attribute data and the environment data. Similar to the first embodiment, in a case where no first perspiration pattern matches the attribute data and the environment data, interpolation processing is performed to identify one first perspiration pattern.

Thereafter, the perspiration pattern identifying section 111 identifies a plurality of first perspiration patterns (two first perspiration patterns in the case of identifying three first perspiration patterns) having characteristics similar to those of the identified one first perspiration pattern. In a case where no first perspiration pattern has the similar characteristics, the perspiration pattern identifying section 111 performs interpolation processing satisfying prescribed conditions to generate first perspiration patterns. In other words, a first perspiration pattern correlated with an attribute value within a prescribed range including the value indicated by the attribute data and/or an environment value within a prescribed range including the value indicated by the environment data is identified. For example, in a case where the acquired attribute data indicates 20 years old and the acquired environment data indicates a temperature of 30° C., the perspiration pattern identifying section 111 generates a first perspiration pattern at a temperature of 29.9° C. or 30.1° C.

The comparing section 112 uses the plural pieces of local perspiration data acquired at the plural times by the perspiration sensor 30 to select one first perspiration pattern among the first perspiration patterns identified by the perspiration pattern identifying section 111. Then, the comparing section 112 identifies a second perspiration pattern corresponding to the first perspiration pattern. In specific, the comparing section 112 compares a fitted curve obtained from the plural pieces of local perspiration data as described above with the first perspiration patterns identified by the perspiration pattern identifying section 111 and selects a first perspiration pattern having the highest level of coincidence. Then, the comparing section 112 identifies a second perspiration pattern corresponding to the selected first perspiration pattern as the second perspiration pattern used for estimation processing at the perspiration state estimating section 113. The comparing section 112 also identifies the time To in the selected first perspiration pattern.

Note that in the above example, the perspiration pattern identifying section 111 identifies a plurality of (in the above example, three of each of) the first and second perspiration patterns correlated with the value indicated by the acquired attribute data and the value indicated by the acquired environment data. No such limitation is intended, and the perspiration pattern identifying section 111 may identify a plurality of only the first perspiration patterns. In this case, the perspiration pattern identifying section 111 selects one first perspiration pattern among the identified first perspiration patterns and then identifies one second perspiration pattern corresponding to the first perspiration pattern among a plurality of second perspiration patterns stored in the storage 12.

The perspiration state estimating section 113 uses the second perspiration pattern and the time To identified by the comparing section 112 to estimate the amount of perspiration on the user's whole body. In the example in FIG. 6, the comparing section 112 obtains a fitted curve for the plural pieces of local perspiration data at the plural times including times corresponding to time Tb and the time To on the horizontal axis of the graph showing the perspiration patterns, and selects the first perspiration pattern P2 as a first perspiration pattern having the highest level of coincidence with the fitted curve. Then, the perspiration state estimating section 113 uses a second perspiration pattern corresponding to the first perspiration pattern P2 to estimate the amount of perspiration on the user's whole body at the time of the highest level of coincidence between the fitted curve and the first perspiration pattern P2 (for example, the time To) (i.e., at the time of the acquisition of the local perspiration data). The perspiration state progression predicting section 114 uses the second perspiration pattern corresponding to the first perspiration pattern P2 to predict progression of the amount of perspiration on the whole body over time after the acquisition of the local perspiration data.

### Perspiration State Estimation Method

Next, a method of estimating the amount of perspiration on the whole body will be described with reference to FIG. 7. FIG. 7 is a flowchart of an example of a method of estimating the amount of perspiration on the whole body according to the present modification. The steps S1, S2, and S4, and the step S6 and subsequent steps in FIG. 7 are similar to those in the first or second embodiment, and descriptions thereof will be omitted.

In the present modification, in S3 illustrated in FIG. 7, the perspiration pattern identifying section 111 identifies a plurality of first perspiration patterns being a target of selection processing at the comparing section 112 among a plurality of perspiration patterns stored in the storage 12, as described above. In S4, the comparing section 112 acquires plural pieces of local perspiration data acquired at a plurality of times by the perspiration sensor 30. In S11, the comparing section 112 obtains a fitted curve for the plural pieces of local perspiration data, fits the fitted curve to the first perspiration patterns identified by the perspiration pattern identifying section 111 to select one first perspiration pattern, and identifies a second perspiration pattern corresponding to the first perspiration pattern (comparing step). The comparing section 112 identifies the time To being time when the local perspiration data is acquired in the first perspiration pattern. Thereafter, the identified time To and second perspiration pattern are used to estimate and predict the amount of perspiration on the whole body and to generate support data.

### Main Advantageous Effect

Even with the same environment data and attribute data (for example, the same temperature, the same age), the number of active sweat glands (the number of sweat glands that are working), the body surface area, the amount of perspiration per sweat gland, and the like may differ between individuals. Thus, even if a perspiration pattern correlated with environment data and/or attribute data is identified, the amount of perspiration on the whole body may not be acquired accurately in some cases.

In the perspiration data estimation device 10A of the present modification, the perspiration pattern identifying section 111 identifies the plural perspiration patterns correlated with the acquired attribute data and environment data. The comparing section 112 uses the plural pieces of local perspiration data to select one first perspiration pattern among the perspiration patterns. Thus, the comparing section 112 can select a perspiration pattern more appropriate for the state (actual condition) of the user. Accordingly, the accuracy in estimating the amount of perspiration on the whole body can be improved.

### Third Embodiment

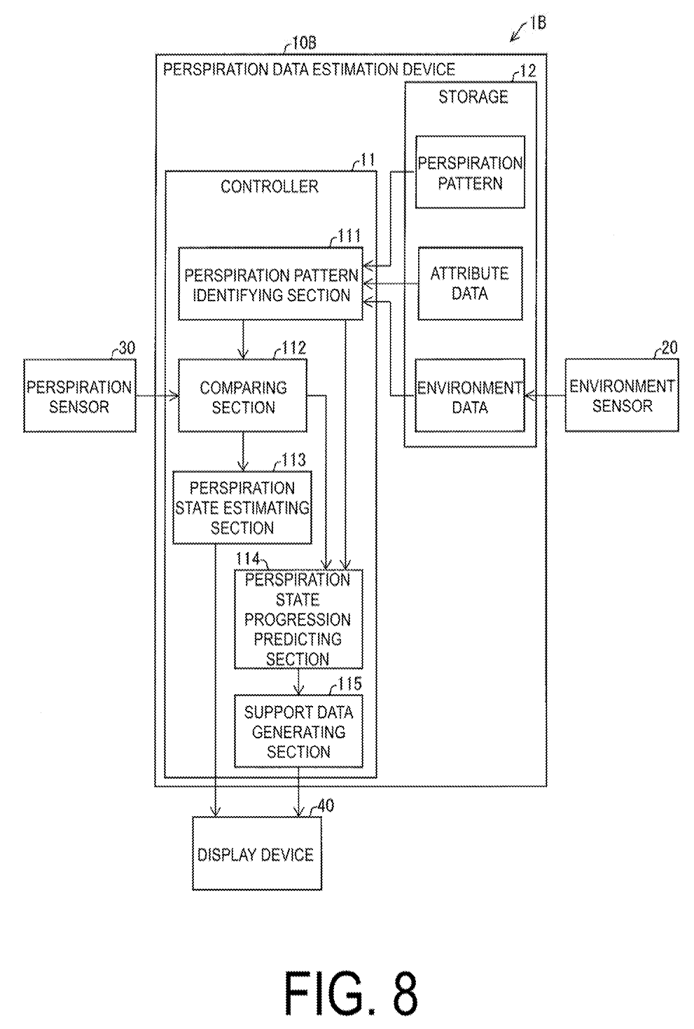

A third embodiment of the disclosure will be described below with reference to FIG. 3 and FIG. 8.

### Configuration of Perspiration State Estimation Device

First, an example of a perspiration data estimation device 10B (perspiration state estimation device) according to the present embodiment will be described with reference to FIG. 8. FIG. 8 is a diagram illustrating an example of a configuration of a user support system 1B according to the present embodiment. The user support system 1B includes the perspiration data estimation device 10B, which differs from the user support system 1 of the first embodiment.

In specific, in the perspiration data estimation device 10B of the present embodiment, the environment sensor 20 acquires environment data at a plurality of times. The comparing section 112 uses a first perspiration pattern identified using the plural pieces of environment data acquired by the environment sensor 20 to perform comparison.

More specifically, in the perspiration data estimation device 10B, environment data acquired at a plurality of times by the environment sensor 20 are temporarily stored in the storage 12. The perspiration pattern identifying section 111 calculates, for example, an average value of values indicated by the plural pieces of environment data acquired at the plural times by the environment sensor 20 (in the case of temperature, an average temperature of a plurality of acquired temperatures). The perspiration pattern identifying section 111 then uses the average value calculated as environment data to identify a perspiration pattern.

Note that an average value of values indicated by plural pieces of environment data acquired in a prescribed time period may be used as a value of environment data in the prescribed time period and afterward. That is, the average value used may be shifted by period after the prescribed period with each of the periods having the same length.

### Living Body State Prediction Method

Next, a method of estimating the amount of perspiration on the whole body will be described with reference to FIG. 3. The step S1, and the step S4, and subsequent steps in FIG. 3 are similar to those in the first embodiment, and descriptions thereof will be omitted.

In S2 in FIG. 3, the environment sensor 20 acquires environment data at a plurality of times and stores the data in the storage 12. In S3, the perspiration pattern identifying section 111 calculates an average value of the values indicated by the plural pieces of environment data stored in the storage 12. Then, the perspiration pattern identifying section 111 uses the calculated average value as a value indicated by environment data to identify first and second perspiration patterns among a plurality of perspiration patterns stored in the storage 12. Thereafter, the first perspiration pattern is compared with the acquired local perspiration data, and the amount of perspiration on the whole body is estimated. Furthermore, the amount of perspiration on the whole body over time is predicted, and support data is generated.

### Main Advantageous Effect

The value indicated by environment data acquired by the environment sensor 20 may have a measurement error due to, for example, variations in manufacturing the environment sensor 20 and the like. In a case where a perspiration pattern is identified using the value indicated by one piece of environment data with a measurement error occurring, a perspiration pattern inappropriate for the comparison may be identified.

The perspiration data estimation device 10B identifies a perspiration pattern in consideration of environment data at a plurality of times, and can thus identify a perspiration pattern while reducing effect of the measurement error, even with the measurement error occurring. In other words, even if there are variations in the acquired environment data, the perspiration pattern used for the comparison can be identified with the variations reduced. Accordingly, the perspiration data estimation device 10B can improve the accuracy in estimating the amount of perspiration on the whole body.

### Fourth Embodiment

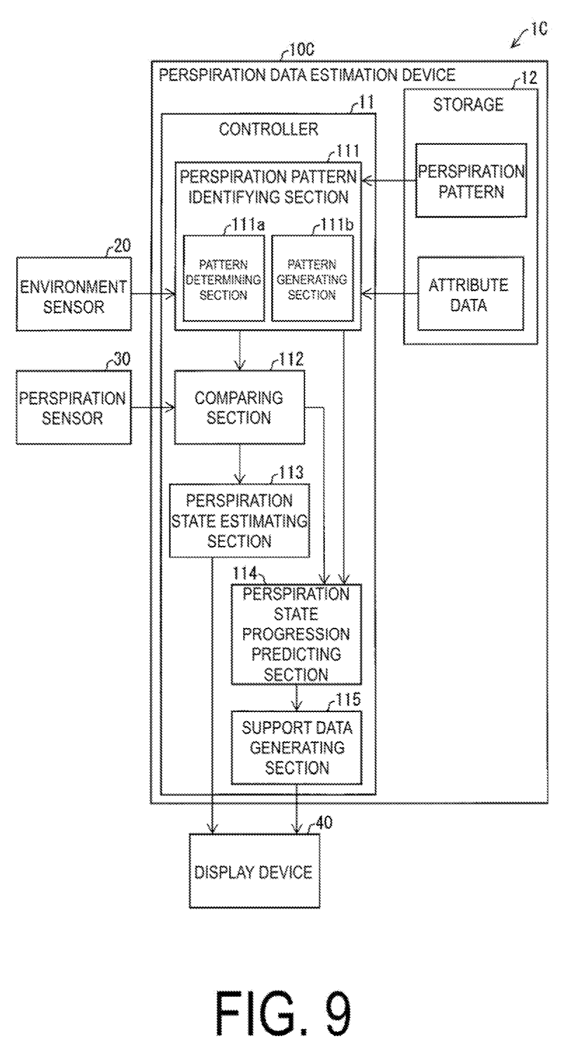

A fourth embodiment of the disclosure will be described below with reference to FIG. 9 to FIG. 11.

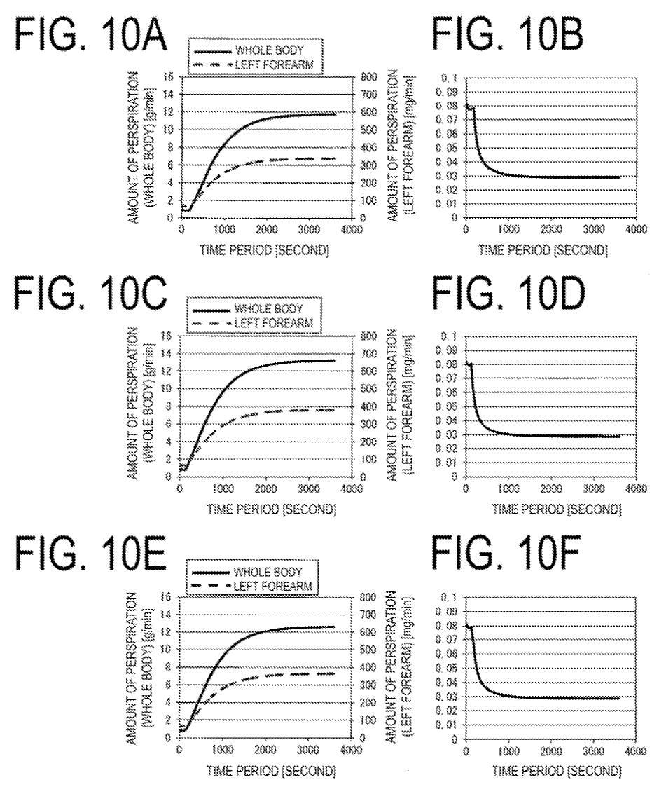

First, an example of a perspiration data estimation device 10C (perspiration state estimation device) according to the present embodiment will be described with reference to FIG. 9 and FIGS. 10A to 10F. FIG. 9 is a diagram illustrating an example of a configuration of a user support system 1C according to the present embodiment. The user support system 1C includes the perspiration data estimation device 10C, which differs from the user support system 1 of the first embodiment.

FIG. 10A is a graph showing a first perspiration pattern (broken line) and a second perspiration pattern (solid line) in the case of a temperature of 20° C. FIG. 10B is a graph showing progression over time of a ratio between the first perspiration pattern and the second perspiration pattern in the case of the temperature of 20° C. FIG. 10C is a graph showing a first perspiration pattern (broken line) and a second perspiration pattern (solid line) in the case of a temperature of 25° C. FIG. 10D is a graph showing progression over time of a ratio between the first perspiration pattern and the second perspiration pattern in the case of the temperature of 25° C.

As illustrated in FIG. 10A and FIG. 10C, the first perspiration pattern and the second perspiration pattern differ between the case of the temperature of 20° C. and the case of the temperature of 25° C. As illustrated in FIG. 10B and FIG. 10D, a ratio between the first perspiration pattern and the second perspiration pattern also differs between the case of the temperature of 20° C. and the case of the temperature of 25° C. This is because, in general, progression of the amount of perspiration differs depending on the temperature (as the temperature is higher, perspiration is caused more rapidly from the start of measuring the amount of perspiration).

In this way, the first perspiration pattern, the second perspiration pattern, and a ratio between these patterns differ depending on the temperature. Thus, the first perspiration pattern, the second perspiration pattern, and a ratio between these patterns in the case of a temperature of, for example, 23° C. differ from those in the cases of the temperatures of 20° C. and 25° C. However, in a case where the storage 12 stores a plurality of perspiration patterns prepared for environment values slightly different from each other, the data size becomes enormous, which is not preferable.

In the perspiration data estimation device 10C, the perspiration pattern identifying section 111 of the controller 11 includes a pattern determining section 111a and a pattern generating section 111b. The pattern determining section 111a determines whether the first perspiration patterns correlated with the predetermined attribute values indicating the attribute include a first perspiration pattern corresponding to the environment data acquired by the environment sensor 20. In a case where the pattern determining section 111a determines that no first perspiration pattern corresponds to the environment data acquired by the environment sensor 20, the pattern generating section 111b uses a plurality of first perspiration patterns correlated with environment values close to the value indicated by the environment data to generate a first perspiration pattern used for comparison at the comparing section 112. The pattern determining section 111a and the pattern generating section 111b perform similar processing for a second perspiration pattern used for prediction of the amount of perspiration on the whole body at the perspiration state estimating section 113 and the perspiration state progression predicting section 114.

FIG. 10E is a graph showing a first perspiration pattern (broken line) and a second perspiration pattern (solid line) generated by the pattern generating section 111b in the case of the temperature of 23° C. A specific example is provided assuming that the value indicated by the environment data is 23° C. and that the storage 12 stores perspiration patterns corresponding to environment values 20° C. and 25° C., which are close to 23° C. In this case, the ratio between (1) a temperature difference between the value 23° C. indicated by the environment data and the environment value 20° C. close to the value and (2) a temperature difference between the value 23° C. indicated by the environment data and the environment value 25° C. close to the value is 3:2. Thus, as illustrated in FIG. 10E, the pattern generating section 111b generates such a point set (locus) that the ratio between the distance from the first perspiration pattern to the point set in the case of the temperature of 20° C. and the distance from the first perspiration pattern to the point set in the case of the temperature of 25° C. is 3:2 at each time (that is, time on the horizontal axis of the graph showing the perspiration patterns), as a first perspiration pattern in the case of the temperature of 23° C. Similarly, the pattern generating section 111b generates such a point set that the ratio between the distance from the second perspiration pattern to the point set in the case of the temperature of 20° C. and the distance from the second perspiration pattern to the point set in the case of the temperature of 25° C. is 3:2 at each time, as a second perspiration pattern in the case of the temperature of 23° C.

FIG. 10F is a graph showing progression over time of a ratio between the first perspiration pattern and the second perspiration pattern in the case of the temperature of 23° C. In a case where the storage 12 stores patterns indicating progression over time of ratios between the first perspiration patterns and the second perspiration patterns in the cases of the temperatures of 20° C. and 25° C., the pattern generating section 111b may generate a pattern indicating progression over time of a ratio between the first perspiration pattern and the second perspiration pattern in the case of the temperature of 23° C. on the basis of the above-described ratio between the distances.

Note that the pattern determining section 111a and the pattern generating section 111b may be provided separate from the perspiration pattern identifying section 111.

The pattern determining section 111a may determine whether the first perspiration patterns correlated with the predetermined environment values indicating the environment include a first perspiration pattern corresponding to the attribute value of the user. In this case, in a case where the pattern determining section 111a determines that no first perspiration pattern corresponds to the attribute data of the user, the pattern generating section 111b uses a plurality of first perspiration patterns correlated with attribute values close to the attribute data of the user to generate a first perspiration pattern used for comparison at the comparing section 112.

An example is provided assuming that the first perspiration patterns correlated with the environment values include first perspiration patterns corresponding to attribute values 20 years old and 25 years old. In this case, in a case where the user is 23 years old, the pattern determining section 111a determines that no first perspiration pattern corresponds to the attribute data of the user. Then, the pattern generating section 111b uses the first perspiration patterns corresponding to the attribute values 20 years old and 25 years old, which are close to the attribute data of the user, to generate a first perspiration pattern used for comparison at the comparing section 112.

Furthermore, in a case where no first perspiration pattern corresponds to either of the attribute data of the user and the environment data, the pattern generating section 111b may generate a first perspiration pattern used for comparison at the comparing section 112.

### Perspiration State Estimation Method

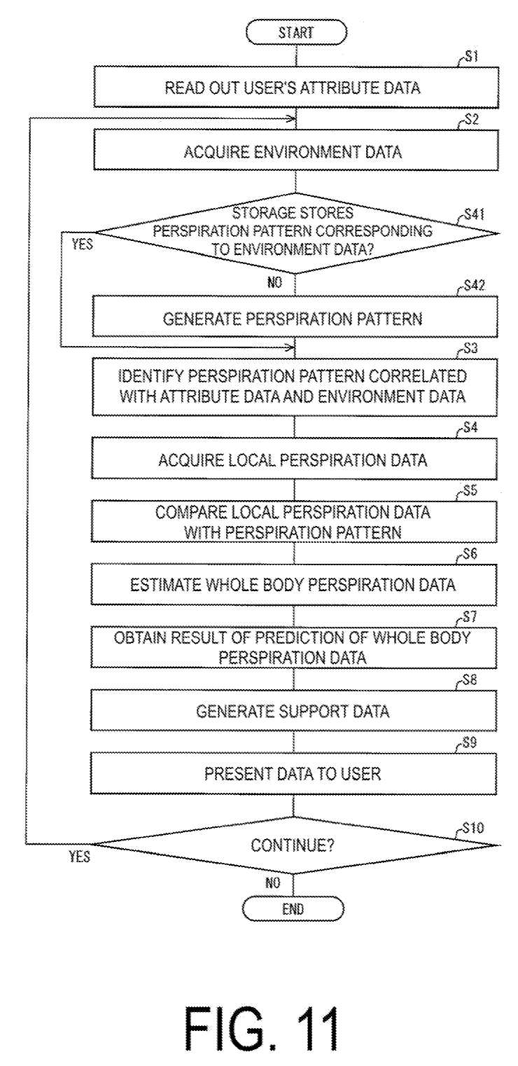

Next, a method of predicting the amount of perspiration on the whole body will be described with reference to FIG. 11. FIG. 11 is a flowchart of an example of a method of predicting the amount of perspiration on the whole body according to the present embodiment. The steps S1 and S2, the step S4, and subsequent steps in FIG. 11 are similar to those in the first embodiment and the like, and descriptions thereof will be omitted.

In S41 in FIG. 11, the pattern determining section 111a determines whether the storage 12 stores a perspiration pattern corresponding to the value indicated by the environment data acquired by the environment sensor 20. If no such perspiration pattern is stored (NO in S41), the pattern generating section 111b generates a perspiration pattern corresponding to the value indicated by the environment data (S42).

In S3, if YES in S41, the perspiration pattern identifying section 111 identifies a first perspiration pattern stored in the storage 12 and corresponding to the environment data as the first perspiration pattern used for comparison at the comparing section 112. On the other hand, if NO in S41, the perspiration pattern identifying section 111 identifies the perspiration pattern generated in S42 as the first perspiration pattern used for comparison at the comparing section 112. Thereafter, the identified perspiration pattern is used to estimate and predict the amount of perspiration on the whole body and to generate support data.

### Main Advantageous Effect

In this way, in a case where the storage 12 stores no perspiration pattern corresponding to the attribute data or environment data, the pattern generating section 111b can generate a perspiration pattern corresponding to the attribute data or environment data. Thus, without preparing a large number of perspiration patterns corresponding to attribute data and environment data in the storage 12, the perspiration data estimation device 10C can accurately estimate the amount of perspiration on the whole body while coping with slight differences between the attribute values or environment values correlated with the prepared perspiration patterns and the value indicated by the actual attribute data or environment data.

### Fifth Embodiment

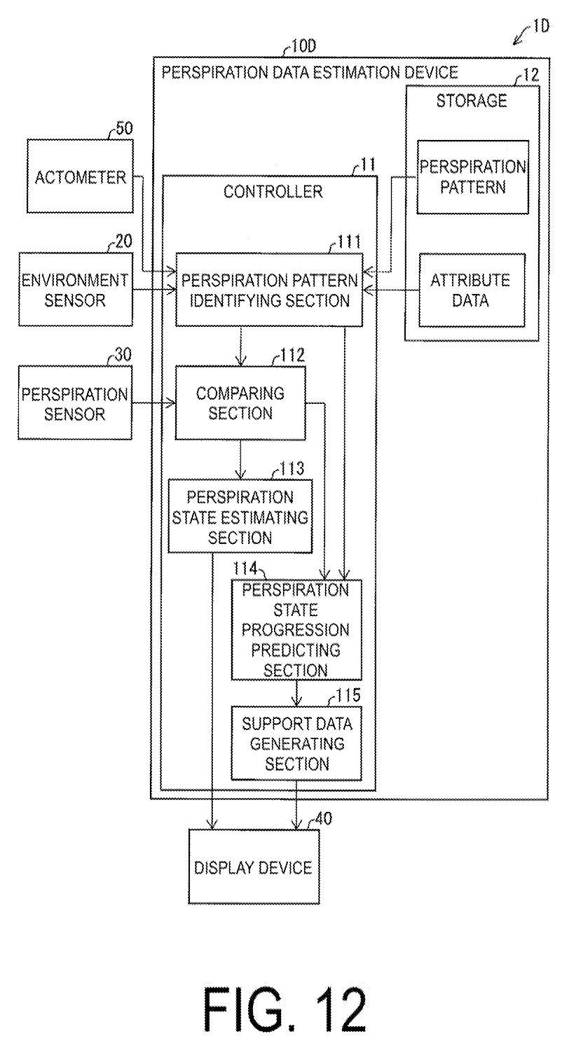

A fifth embodiment of the disclosure will be described below with reference to FIG. 12 to FIG. 14.

First, an example of a perspiration data estimation device 10D (perspiration state estimation device) according to the present embodiment will be described with reference to FIG. 12. FIG. 12 is a diagram illustrating an example of a configuration of a user support system 1D according to the present embodiment. The user support system 1D includes the perspiration data estimation device 10D and an actometer 50 (activity data acquiring unit), which differs from the user support system 1 of the first embodiment.

The actometer 50 is connected to the perspiration data estimation device 10D in a communicable manner and acquires activity data indicating an activity state of the user. The actometer 50 transmits the acquired activity data to the perspiration data estimation device 10D.

The actometer 50 is equipped with an acceleration sensor and calculates the amount of exercise, calorie consumption, or the like of the user on the basis of acceleration caused by a motion of the user and detected by the acceleration sensor. In the present embodiment, the actometer 50 converts the amount of exercise, calorie consumption, or the like into a metabolic equivalent (MET) being an index of the intensity of physical activities (the amount of activities) to calculate the MET as activity data.

The MET is an index of the amount of activities of a living body indicating how many times more the energy is consumed than one MET, which is defined as the energy taking to be at rest. That is, the MET value gets higher as the user exercise more vigorously.

Note that the activity data acquiring unit acquiring activity data is not limited to the actometer 50 and may be, for example, a pedometer. In the case of a pedometer, a walking speed, a time period taken for one step, or the like is calculated on the basis of acceleration detected by an acceleration sensor mounted in the pedometer. Then, the pedometer converts the walking speed, the time period taken for one step, or the like into MET to acquire activity data. That is, the activity data acquiring unit may have any configuration, as long as the unit includes a sensor capable of detecting a motion of the user (such as an acceleration sensor) and can acquire activity data.

In the present embodiment, MET is described as an example of the activity data; however, no such limitation is intended. The activity data may indicate the amount of exercise or calorie consumption of the user acquired by the actometer 50, or the walking speed, the time period taken for one step, or the like acquired by the pedometer. The perspiration pattern identifying section 111 may calculate MET. In this case, the above-described data acquired by the actometer 50 or the pedometer is transmitted to the perspiration pattern identifying section 111.

The actometer 50 may be equipped with, for example, a pulsimeter or a heart rate meter, in addition to the acceleration sensor, and may acquire a measurement result from the meter as the activity data.

In the perspiration data estimation device 10D, the perspiration patterns stored in the storage 12 are correlated with not only the environment data and/or the attribute data but also a plurality of predetermined activity values indicating an activity state of the user (in the present embodiment, METs indicating the amounts of activities).

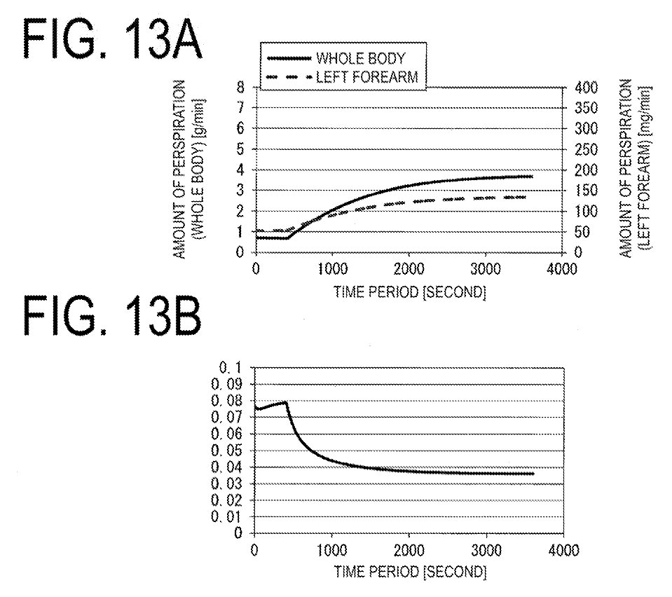

FIG. 13A is a diagram illustrating an example of perspiration patterns in the case of a prescribed MET value. FIG. 13B is a diagram illustrating a ratio between a first perspiration pattern and a second perspiration pattern on the basis of the perspiration patterns illustrated in FIG. 13A.

The perspiration patterns illustrated in FIG. 2A and the ratio between the first perspiration pattern and the second perspiration pattern illustrated in FIG. 2B can be considered as those in the case of another prescribed MET value greater than the above-described prescribed MET value. As illustrated in FIG. 13A and FIG. 13B, the perspiration patterns and the ratio between the first perspiration pattern and the second perspiration pattern in the case of the prescribed MET value differ significantly from, for example, those in the case illustrated in FIG. 2A and FIG. 2B. In this way, similar to the environment data and the attribute data, the activity data affects the perspiration patterns. Accordingly, by correlating the perspiration patterns with the activity data, the accuracy in estimating the amount of perspiration on the whole body can be improved.

The perspiration pattern identifying section 111 also uses the activity data acquired by the actometer 50 to identify a perspiration pattern used for comparison at the comparing section 112 among a plurality of perspiration patterns also correlated with the activity values. In other words, the perspiration pattern used for comparison at the comparing section 112 is also correlated with the activity data acquired by the actometer 50.

Note that, similar to the first embodiment, a mathematical expression for calculating a perspiration pattern may be prepared in the storage 12, and the perspiration pattern identifying section 111 may insert (1) a value indicated by the attribute data and/or the environment data and (2) a value indicated by the activity data into the mathematical expression to identify a perspiration pattern used by the comparing section 112.

Similar to the fourth embodiment, the perspiration data estimation device 10D may include a pattern determining section and a pattern generating section for generating a perspiration pattern in consideration of a change, if made, in the amount of activities over time. In this case, for example, the pattern determining section 111a determines whether a plurality of first perspiration patterns correlated with the activity values include a first perspiration pattern corresponding to the activity data acquired by the actometer 50. In a case where it is determined that no such first perspiration pattern is included, the pattern generating section 111b uses a plurality of first perspiration patterns correlated with activity values close to the value indicated by the activity data to generate a first perspiration pattern used for comparison at the comparing section 112.

Furthermore, in a case where no first perspiration pattern corresponds to two types or more of the attribute data, the environment data, and the activity data, the pattern generating section 111b may generate a first perspiration pattern used for comparison at the comparing section 112.

### Living Body State Prediction Method

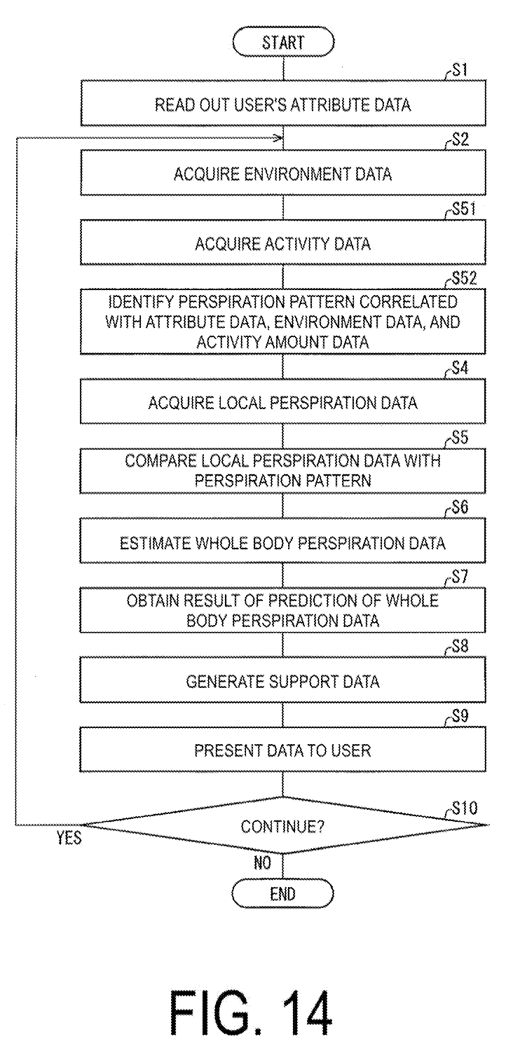

Next, a method of predicting the amount of perspiration on the whole body will be described with reference to FIG. 14. FIG. 14 is a flowchart of an example of a method of predicting the amount of perspiration according to the present embodiment. The steps 51 and S2, the step S4, and subsequent steps in FIG. 14 are similar to those in the first embodiment, and descriptions thereof will be omitted.

In S51 in FIG. 14, the actometer 50 acquires activity data. The actometer 50 may acquire activity data and transmit the data to the perspiration pattern identifying section 111 in response to a request from the perspiration pattern identifying section 111 or may transmit activity data nearest to the time of the request among accumulated activity data to the perspiration pattern identifying section 111, for example.

The perspiration pattern identifying section 111 identifies a perspiration pattern correlated with (1) the read out attribute data, (2) the environment data acquired from the environment sensor 20, and (3) the activity data acquired from the actometer 50 among a plurality of perspiration patterns stored in the storage 12, as the perspiration pattern used by the comparing section 112 (S52). Thereafter, the identified first perspiration pattern is compared with the acquired local perspiration data, and a result of this comparison and the identified second perspiration pattern are used to estimate and predict the amount of perspiration on the whole body and to generate support data.

Note that (1) the steps S2, S51, and S52 and (2) the step S4 may be performed simultaneously, or the steps (1) may be performed after the step (2). The steps S2 and S51 may be performed simultaneously or in reverse order.

### Main Advantageous Effect

In the perspiration data estimation device 10D, the comparing section 112 performs comparison using the perspiration pattern in consideration of an activity state of the user, so that the accuracy in estimating the amount of perspiration on the whole body can be improved.

### Sixth Embodiment

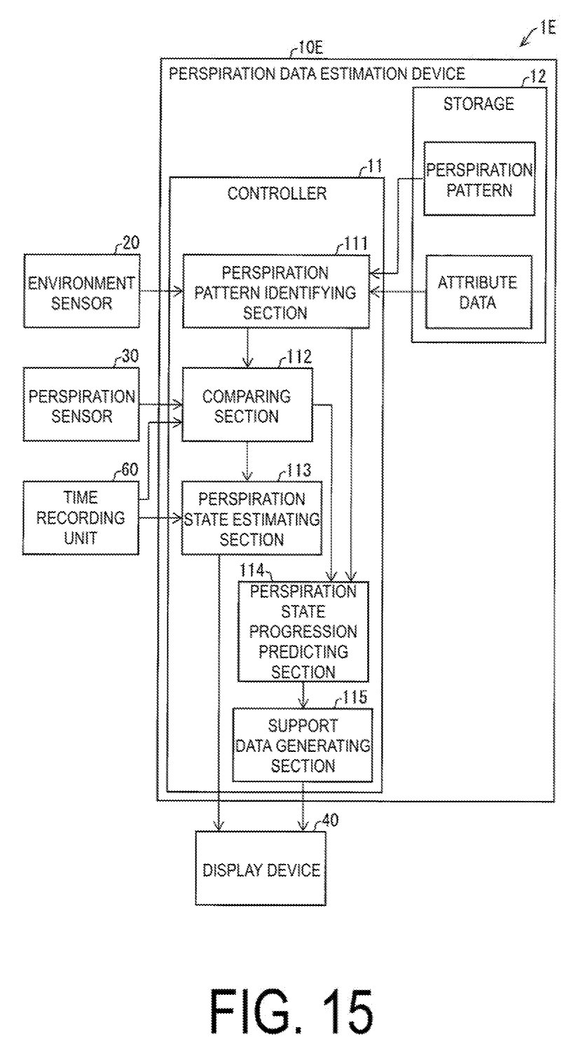

A sixth embodiment of the disclosure will be described below with reference to FIG. 15 to FIG. 17.

First, an example of a perspiration data estimation device 10E (perspiration state estimation device) according to the present embodiment will be described with reference to FIG. 15. FIG. 15 is a diagram illustrating an example of a configuration of a user support system 1E according to the present embodiment. The user support system 1E includes the perspiration data estimation device 10E and a time recording unit 60, which differs from the user support system 1 of the first embodiment.

The time recording unit 60 is connected to the perspiration data estimation device 10E in a communicable manner and records time. The time recording unit 60 transmits recorded time data indicating the recorded time to the perspiration data estimation device 10E.

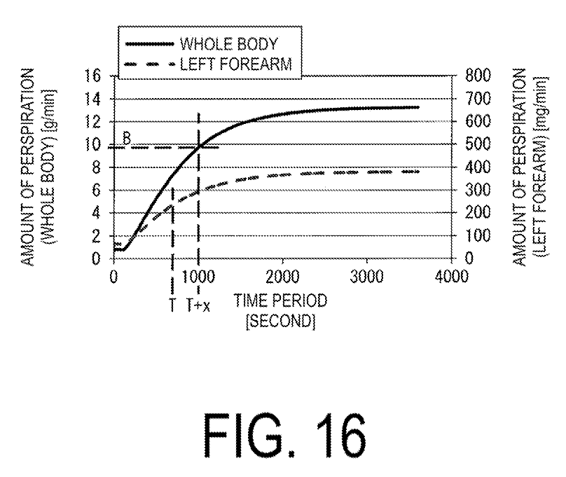

FIG. 16 is a diagram for describing estimation of the amount of perspiration on the whole body in the perspiration data estimation device 10E. First, similar to the perspiration data estimation device 10 of the first embodiment, the comparing section 112 of the perspiration data estimation device 10E acquires the value indicated by the local perspiration data at least once and identifies time T corresponding to the value in the first perspiration pattern. At this time, the comparing section 112 acquires recorded time data indicating actual time when the time T is identified from the time recording unit 60 and stores the data in the storage 12.

When the time T is identified, the perspiration data estimation device 10E can estimate the amount of perspiration on the whole body without acquiring the local perspiration data. To estimate the amount of perspiration on the whole body, the perspiration state estimating section 113 estimates the amount of perspiration on the whole body at the moment when a prescribed time period is elapsed from the time T identified by the comparing section 112. In specific, the perspiration state estimating section 113 acquires the recorded time data indicating the time of the estimation (for example, time recorded after actual time when the time T is identified) from the time recording unit 60. By identifying a time period elapsed from the actual time corresponding to the time T to the time indicated by the recorded time data, the amount B of perspiration on the whole body at the moment when the prescribed time period x is elapsed from the time T (i.e., time T+x illustrated in FIG. 16) in the second perspiration pattern is estimated.

### Living Body State Prediction Method

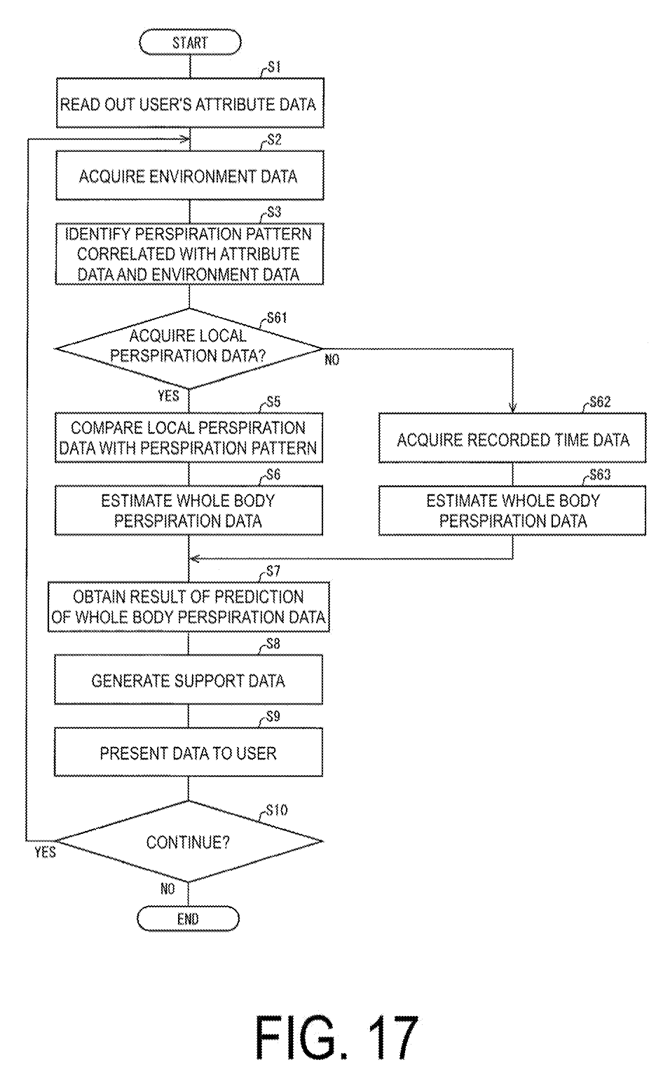

Next, a method of estimating the amount of perspiration on the whole body will be described with reference to FIG. 17. FIG. 17 is a flowchart of an example of a method of estimating the amount of perspiration according to the present embodiment. The steps S1 to S3, and the step S5 and subsequent steps in FIG. 17 are similar to those in the first embodiment, and descriptions thereof will be omitted.

After the step S3, a data acquisition determining section (not illustrated) of the controller 11 determines whether the perspiration sensor 30 acquires local perspiration data (S61). If local perspiration data is acquired (YES in S61), the comparing section 112 performs comparison to identify time T corresponding to the value indicated by the local perspiration data in the first perspiration pattern through processes similar to those in the first embodiment and the like. Thereafter, the amount of perspiration on the whole body is estimated, progression of the amount of perspiration on the whole body is predicted, and support data is generated. In this case, in S5, the comparing section 112 acquires recorded time data (data indicating actual time corresponding to the time T) is acquired from the time recording unit 60. In the present embodiment, the step S6 may be omitted.

If no local perspiration data is acquired (NO in S61), the time recording unit 60 records time when the prescribed time period is elapsed from the time T (time corresponding to the time T+x). Then, the perspiration state estimating section 113 acquires the recorded time data indicating the time from the time recording unit 60 (S62). The time recording unit 60 acquires the recorded time data and transmits the data to the perspiration state estimating section 113 in response to a request from the perspiration state estimating section 113, for example. Then, the perspiration state estimating section 113 estimates the amount of perspiration on the whole body at the time T+x on the basis of the time T identified in S5 and the recorded time data acquired from the time recording unit 60 in S62 and indicating the time (S63). Thereafter, progression of the amount of perspiration on the whole body is predicted, and support data is generated.

Note that, if the time T is not identified, that is, if the path of YES is never taken in S61, the steps S62 and S63 (i.e., the steps of estimating the amount of perspiration on the whole body) are not performed. Thus, if the determination in S61 results in NO without the time T identified, the data acquisition determining section skips the step S62 and subsequent steps. In this case, the step to be performed subsequently may be, for example, S10.

After the time T is identified once, local perspiration data may be acquired from the perspiration sensor 30 by the prescribed time period and compared with the first perspiration pattern to identify time T again.

In the above description, in S61, the data acquisition determining section determines whether the perspiration sensor 30 acquires local perspiration data, and if local perspiration data is acquired, the comparison is performed. However, if the comparison is performed by the prescribed time period, the perspiration sensor 30 may acquire local perspiration data between time of comparison and time of subsequent comparison. In this case, the data acquisition determining section may have a function to determine whether the acquired local perspiration data is used for comparison, depending on the time. In this case, a time interval to the subsequent comparison at the comparing section 112 may be longer than a time interval to subsequent acquisition of local perspiration data at the perspiration sensor 30.

### Main Advantageous Effect

With the perspiration data estimation device 10E, once the perspiration sensor 30 acquires the local perspiration data and the time T is identified, the time interval to the subsequent acquisition of the local perspiration data at the perspiration sensor 30 can be longer than a time interval to subsequent estimation of the amount of perspiration on the whole body at the perspiration state estimating section 113. Alternatively, without the perspiration sensor 30 acquiring the local perspiration data again, the perspiration state estimating section 113 can estimate the amount of perspiration on the whole body. Accordingly, a load on the perspiration data estimation device 10E due to the step of acquiring the local perspiration data at the perspiration sensor 30 can be reduced.

### Seventh Embodiment

In the above-described embodiments, the perspiration pattern is preliminarily stored in the storage 12 and read out by the perspiration pattern identifying section 111. The perspiration pattern may be updated using a prescribed database. A perspiration pattern for a condition (for example, a temperature or an attribute) not correlated with perspiration estimation data stored in the storage 12 may be newly added using a prescribed database. Such a database may be prepared, for example, in a cloud environment.

The above-described update or addition enables the perspiration state estimation device of the present embodiment to estimate the amount of perspiration on the whole body on the basis of perspiration patterns corresponding to more accurate or more various environment values. Accordingly, the perspiration state estimation device can improve the accuracy in estimating amount of perspiration on the whole body.

Furthermore, the above-described addition can decrease the number of times of interpolation processing and can thus reduce a processing road of the controller 11. Moreover, with the database prepared in a cloud environment, the storage capacity of the storage 12 can be efficiently used.

### Implementation Example by Software

A control block (in particular, the controller 11) of the perspiration data estimation devices 10, 10A, 10B, 10C, 10D, and 10E may be realized by a logic circuit (hardware) formed in an integrated circuit (IC chip) and the like, or by software by using a Central Processing Unit (CPU).

In the latter configuration, the perspiration data estimation devices 10 and 10A to 10E each include a CPU for executing instructions of a program which is software for implementing each function, a Read Only Memory (ROM) or a storage device (each of these is referred to as a “recording medium”) in which the program and various types of data are recorded in a computer-readable (or CPU-readable) manner, a Random Access Memory (RAM) in which the program is loaded, and the like. Then, the computer (or CPU) reads the program from the recording medium and executes the program to achieve the object of an aspect of the disclosure. As the recording medium, a “non-transitory tangible medium”, such as a tape, a disk, a card, a semiconductor memory, and a programmable logic circuit may be used. Further, the program may be supplied to the computer via any transmission medium (a communication network, a broadcast wave, or the like) able to transmit the program. Note that an aspect of the disclosure may be implemented in a form of data signal embedded in a carrier wave, which is embodied by electronic transmission of the program.

### Supplement

A perspiration state estimation device (perspiration data estimation device 10, 10A to 10E) according to a first aspect of the disclosure is connected to a local perspiration data acquiring unit (perspiration sensor 30) and an environment data acquiring unit (environment sensor 20) in a communicable manner, the local perspiration data acquiring unit being configured to acquire local perspiration data indicating a perspiration state of a local part of a living body, the environment data acquiring unit being configured to acquire environment data indicating a state of an environment where the living body is present, and includes: a comparing section (112) configured to compare (1) the local perspiration data acquired by the local perspiration data acquiring unit with (2) a first perspiration pattern correlated with at least either of attribute data indicating an attribute of the living body and the environment data acquired by the environment data acquiring unit, the first perspiration pattern indicating progression of the perspiration state of the local part over time; and an estimating section (perspiration state estimating section 113) configured to estimate a perspiration state of a site of the living body on a basis of a result of the comparison at the comparing section and a progression relating pattern indicating a second perspiration pattern indicating progression of the perspiration state of the site over time or a progression relating pattern indicating a relationship between the first perspiration pattern and the second perspiration pattern, the site including at least a part other than the local part.

With the above configuration, the comparing section compares the local perspiration data acquired by the local perspiration data acquiring unit with the first perspiration pattern. The estimating section estimates perspiration data of the site of the living body including at least a part other than the local part of the living body of which the local perspiration data is acquired by the local perspiration data acquiring unit, on the basis of a result of the comparison at the comparing section and the progression relating pattern.

That is, when the perspiration state of the site of the living body is estimated from the perspiration state of the local part, the perspiration state estimation device uses the first perspiration pattern indicating progression of the perspiration state of the local part over time and estimation relating pattern relating to progression of the perspiration state of the site of the living body over time to estimate the perspiration state of the site of the living body. The perspiration state estimation device uses these two patterns indicating states over time, and can thus estimate the perspiration state of the site of the living body in consideration of the perspiration state different depending on the local part (for example, the timing of starting perspiration and/or the amount of perspiration). Accordingly, the perspiration state estimation device can accurately estimate the perspiration state of the site of the living body on the basis of the perspiration state of the local part of the living body.

In a perspiration state estimation device according to a second aspect of the disclosure having the configuration of the first aspect, the estimating section is preferably configured to estimate the perspiration state of the site on a basis of (1) the second perspiration pattern being the progression relating pattern and (2) time (To) identified through the comparison at the comparing section, the time corresponding to a value indicated by the local perspiration data in the first perspiration pattern.

With the above configuration, the estimating section can estimate the perspiration state of the site of the living body at the time identified by the comparing section in the second perspiration pattern. This enables accurate estimation of the perspiration state of the site of the living body at the time identified by the comparing section.

A perspiration state estimation device according to a third aspect of the disclosure having the configuration of the first or second aspect, preferably further includes an identifying section (perspiration pattern identifying section 111) configured to identify at least either of (1) a first perspiration pattern corresponding to the attribute data among a plurality of first perspiration patterns correlated with a plurality of predetermined attribute values indicating the attribute and (2) a first perspiration pattern corresponding to the environment data among a plurality of first perspiration patterns correlated with a plurality of predetermined environment values indicating a prescribed environment state, and the comparing section is preferably configured to perform the comparison using the first perspiration pattern identified by the identifying section.

With the above configuration, only by acquiring the attribute data and the environment data, the first perspiration pattern used for the comparison can be identified among the prepared first perspiration patterns.

In a perspiration state estimation device (perspiration data estimation device 10A) according to a fourth aspect of the disclosure having the configuration of any one of the first to third aspects, the local perspiration data acquiring unit is preferably configured to acquire the local perspiration data at a plurality of times, and the comparing section is preferably configured to perform comparison using plural pieces of the local perspiration data acquired by the local perspiration data acquiring unit.

With the above configuration, even if there are variation in the acquired local perspiration data, effect of the variations that may be exerted on the comparison can be reduced. Accordingly, the accuracy in estimating the perspiration state of the site of the living body can be improved.

In a perspiration state estimation device according to a fifth aspect the disclosure having the configuration of the fourth aspect, the first perspiration pattern preferably includes a plurality of first perspiration patterns identified and used for comparison at the comparing section, the comparing section is preferably configured to use the plural pieces of local perspiration data to select a first perspiration pattern from the first perspiration patterns identified, and the estimating section is preferably configured to estimate the perspiration state of the site of the living body using the progression relating pattern corresponding to the first perspiration pattern selected by the comparing section.

With the above configuration, the estimating section estimates the perspiration state of the site of the living body using the progression relating pattern corresponding to the first perspiration pattern more appropriate for the state of the user. Accordingly, the accuracy in estimating the perspiration state of the site of the living body can be improved.

In a perspiration state estimation device (perspiration data estimation device 10B) according to a sixth aspect of the disclosure having the configuration of any one of the first to fifth aspects, the environment data acquiring unit is preferably configured to acquire the environment data at a plurality of times, and the comparing section is preferably configured to perform comparison using a first perspiration pattern identified using plural pieces of the environment data acquired by the environment data acquiring unit.

With the above configuration, even if there are variations in the acquired environment data, the comparing section can perform the comparison using the first perspiration pattern with the variations reduced.

In a perspiration state estimation device (perspiration data estimation device 10D) according to a seventh aspect of the disclosure having the configuration of any one of the first to sixth aspects, the perspiration state estimation device preferably further includes an activity data acquiring unit (actometer 50) configured to acquire activity data indicating an activity state of the living body, and the comparing section is preferably configured to perform comparison using a first perspiration pattern further correlated with the activity data acquired by the activity data acquiring unit.

With the above configuration, the comparing section performs comparison using the first perspiration pattern in consideration of an activity state of the living body, so that the accuracy in estimating the perspiration state can be improved.

A perspiration state estimation device (perspiration data estimation device 10C) according to an eighth aspect of the disclosure having the configuration of any one of the first to sixth aspects, preferably further includes: a pattern determining section (111a) configured to determine whether at least either of (1) a plurality of first perspiration patterns correlated with a plurality of predetermined attribute values indicating the attribute and (2) a plurality of first perspiration patterns correlated with a plurality of predetermined environment values indicating a prescribed environment state include a first perspiration pattern corresponding to the attribute data of the living body or the environment data acquired by the environment data acquiring unit; and a pattern generating section (111b) configured to, upon determination of no first perspiration pattern corresponding to the attribute data or the environment data at the pattern determining section, use a plurality of first perspiration patterns correlated with at least either of attribute values close to a value indicated by the attribute data and environment values close to a value indicated by the environment data to generate a first perspiration pattern used for comparison at the comparing section.

With the above configuration, in a case where no first perspiration pattern corresponds to the attribute data or the environment data, the pattern generating section generates a first perspiration pattern used for comparison at the comparing section. Thus, even if no first perspiration pattern corresponds to the attribute data or the acquired environment data, the perspiration state can be accurately estimated. Furthermore, the first perspiration pattern is generated as described above, so that without preparing a large number of perspiration patterns corresponding to attribute data and environment data, the perspiration state can be accurately estimated while slight differences between the attribute values or environment values correlated with prepared perspiration patterns and the value indicated by the actual attribute data or environment data are coped with.

A perspiration state estimation device according to a ninth aspect of the disclosure having the configuration of the seventh aspect, preferably further includes: a pattern determining section (111a) configured to determine whether at least any one of (1) a plurality of first perspiration patterns correlated with a plurality of predetermined attribute values indicating the attribute, (2) a plurality of first perspiration patterns correlated with a plurality of predetermined environment values indicating a prescribed environment state, and (3) a plurality of first perspiration patterns correlated with a plurality of predetermined activity values indicating a prescribed activity state of the living body include a first perspiration pattern corresponding to the attribute data indicating the attribute of the living body, the environment data acquired by the environment data acquiring unit, or the activity data acquired by the activity data acquiring unit; and a pattern generating section (111b) configured to, upon determination of no first perspiration pattern corresponding to the attribute data, the environment data, or the activity data at the pattern determining section, use a plurality of first perspiration patterns correlated with at least any one of a set of attribute values close to a value indicated by the attribute data, a set of environment values close to a value indicated by the environment data, or a set of activity values close to a value indicated by the activity data to generate a first perspiration pattern used for comparison at the comparing section.

With the above configuration, in a case where no first perspiration pattern corresponds to the attribute data, the environment data, or the activity data, the pattern generating section generates a first perspiration pattern used for comparison at the comparing section. Thus, even if no first perspiration pattern corresponds to the attribute data, the acquired environment data, or the acquired activity data, the perspiration state can be accurately estimated. Furthermore, the first perspiration pattern is generated as described above, so that without preparing a large number of perspiration patterns corresponding to attribute data, environment data, and activity data, the perspiration state can be accurately estimated while slight differences between the attribute values, environment values, or activity values correlated with prepared perspiration patterns and the value indicated by the actual attribute data, environment data, or activity data are coped with.

In a perspiration state estimation device (perspiration data estimation device 10E) according to a tenth aspect of the disclosure having the configuration of any one of the first to ninth aspects, the comparing section is preferably configured to perform comparison to identify time corresponding to a value indicated by the local perspiration data in the first perspiration pattern, and the estimating section is preferably configured to estimate the perspiration state of the site of the living body at a moment when a prescribed time period is elapsed from the time identified as a result of the comparison by the comparing section.

With the above configuration, the perspiration state at the moment when the prescribed time period is elapsed from the time when the local perspiration data is acquired can be estimated on the basis of the time identified by the comparing section and the prescribed time period, that is, without acquiring the local perspiration data. Thus, a time interval of acquiring the local perspiration data can be longer than a time interval of estimating the perspiration state. Accordingly, a load due to the process of acquiring the local perspiration data can be reduced.

In a perspiration state estimation device according to an eleventh aspect of the disclosure having the configuration of any one the first to tenth aspects, the environment data acquiring unit is preferably configured to acquire data indicating at least either of temperature and humidity of the environment, as the environment data.

With the above configuration, the perspiration state can be estimated using a first perspiration pattern correlated with at least either of the temperature and humidity of the environment.

In a perspiration state estimation device according to a twelfth aspect of the disclosure having the configuration of any one of the first to eleventh aspects, the attribute preferably includes at least any of body-build, age, sex, and cloth information of the living body.

With the above configuration, the perspiration state can be estimated using a first perspiration pattern correlated with at least any one of the body-build, age, sex, and cloth information of the user.

A perspiration state estimation method according to a thirteenth aspect of the disclosure includes: a local perspiration data acquiring step of acquiring local perspiration data indicating a perspiration state of a local part of a living body; an environment data acquiring step of acquiring environment data indicating a state of an environment where the living body is present; a comparing step of comparing (1) the local perspiration data acquired in the local perspiration data acquiring step with (2) a first perspiration pattern correlated with at least either of attribute data indicating an attribute of the living body and the environment data acquired in the environment data acquiring step, the first perspiration pattern indicating progression of the perspiration state of the local part over time; and an estimating step of estimating a perspiration state of a site of the living body on a basis of a result of the comparison in the comparing step and a progression relating pattern indicating a second perspiration pattern indicating progression of the perspiration state of the site over time or a progression relating pattern indicating a relationship between the first perspiration pattern and the second perspiration pattern, the site including at least a part other than the local part.

With the above configuration, advantageous effects similar to those of the first aspect are exhibited.

A perspiration state estimation program according to a fourteenth aspect of the disclosure causes a computer to function as the perspiration state estimation device according to the first aspect, and the perspiration state estimation program is configured to cause a computer to function as the comparing section and the estimating section.

The perspiration state estimation device according to each aspect of the disclosure may be realized by a computer. In this case, the perspiration state estimation program realizing the perspiration state estimation device by a computer by operating the computer as each component (software module) included in the perspiration state estimation device, and a computer-readable recording medium storing the perspiration state estimation program fall within the scope of an aspect of the disclosure.

An aspect of the disclosure is not limited to each of the above-described embodiments. Various modifications can be made within the scope of the claims. An embodiment obtained by appropriately combining technical elements each disclosed in different embodiments falls also within the technical scope of an aspect of the disclosure. Furthermore, technical elements disclosed in the respective embodiments may be combined to provide a new technical feature.

### CROSS REFERENCE TO RELATED APPLICATIONS

This application claims priority to JP 2016-112254, filed on Jun. 3, 2016, the entire content of which is incorporated herein by reference.

### REFERENCE SIGNS LIST

10, 10A, 10B, 10C, 10D, 10E Perspiration data estimation device (Perspiration state estimation device)
111 Perspiration pattern identifying section (Identifying section)
111a Pattern determining section
111b Pattern generating section
112 Comparing section
113 Perspiration state estimating section (Estimating section)
20 Environment sensor (Environment data acquiring unit)
30 Perspiration sensor (Local perspiration data acquiring unit)
50 Actometer (Activity data acquiring unit)

1. A perspiration state estimation device capable of being connected to a local perspiration data acquiring unit and an environment data acquiring unit in a communicable manner, the local perspiration data acquiring unit being configured to acquire local perspiration data indicating a perspiration state of a local part of a living body, the environment data acquiring unit being configured to acquire environment data indicating a state of an environment where the living body is present, the perspiration state estimation device comprising:
a comparing section configured to compare (1) the local perspiration data acquired by the local perspiration data acquiring unit with (2) a first perspiration pattern correlated with at least either of attribute data indicating an attribute of the living body and the environment data acquired by the environment data acquiring unit, the first perspiration pattern indicating progression of the perspiration state of the local part over time; and
an estimating section configured to estimate a perspiration state of a site of the living body on a basis of a result of the comparison at the comparing section and a progression relating pattern indicating a second perspiration pattern indicating progression of the perspiration state of the site over time or a progression relating pattern indicating a relationship between the first perspiration pattern and the second perspiration pattern, the site including at least a part other than the local part.

2. The perspiration state estimation device according to claim 1,
wherein the estimating section is configured to estimate the perspiration state of the site on a basis of (1) the second perspiration pattern being the progression relating pattern and (2) time identified through the comparison at the comparing section, the time corresponding to a value indicated by the local perspiration data in the first perspiration pattern.

3. The perspiration state estimation device according to claim 1,
wherein the perspiration state estimation device further includes an identifying section configured to identify at least either of (1) a first perspiration pattern corresponding to the attribute data among a plurality of first perspiration patterns correlated with a plurality of predetermined attribute values indicating the attribute and (2) a first perspiration pattern corresponding to the environment data among a plurality of first perspiration patterns correlated with a plurality of predetermined environment values indicating a prescribed environment state, and
the comparing section is configured to perform the comparison using the first perspiration pattern identified by the identifying section.

4. The perspiration state estimation device according to claim 1,
wherein the local perspiration data acquiring unit is configured to acquire the local perspiration data at a plurality of times, and
the comparing section is configured to perform comparison using plural pieces of the local perspiration data acquired by the local perspiration data acquiring unit.

5. The perspiration state estimation device according to claim 4,
wherein the first perspiration pattern includes a plurality of first perspiration patterns identified and used for comparison at the comparing section,
the comparing section is configured to use the plural pieces of local perspiration data to select a first perspiration pattern from the first perspiration patterns identified, and
the estimating section is configured to estimate the perspiration state of the site of the living body using the progression relating pattern corresponding to the first perspiration pattern selected by the comparing section.

6. The perspiration state estimation device according to claim 1,
wherein the environment data acquiring unit is configured to acquire the environment data at a plurality of times, and
the comparing section is configured to perform comparison using a first perspiration pattern identified using plural pieces of the environment data acquired by the environment data acquiring unit.

7. The perspiration state estimation device according to claim 1,
wherein the perspiration state estimation device further includes an activity data acquiring unit configured to acquire activity data indicating an activity state of the living body, and
the comparing section is configured to perform comparison using a first perspiration pattern further correlated with the activity data acquired by the activity data acquiring unit.

8. The perspiration state estimation device according to claim 1, further comprising:
a pattern determining section configured to determine whether at least either of (1) a plurality of first perspiration patterns correlated with a plurality of predetermined attribute values indicating the attribute and (2) a plurality of first perspiration patterns correlated with a plurality of predetermined environment values indicating a prescribed environment state include a first perspiration pattern corresponding to the attribute data of the living body or the environment data acquired by the environment data acquiring unit; and
a pattern generating section configured to, upon determination of no first perspiration pattern corresponding to the attribute data or the environment data at the pattern determining section, use a plurality of first perspiration patterns correlated with at least either of attribute values close to a value indicated by the attribute data and environment values close to a value indicated by the environment data to generate a first perspiration pattern used for comparison at the comparing section.

9. The perspiration state estimation device according to claim 7, further comprising:
a pattern determining section configured to determine whether at least any one of (1) a plurality of first perspiration patterns correlated with a plurality of predetermined attribute values indicating the attribute, (2) a plurality of first perspiration patterns correlated with a plurality of predetermined environment values indicating a prescribed environment state, and (3) a plurality of first perspiration patterns correlated with a plurality of predetermined activity values indicating a prescribed activity state of the living body include a first perspiration pattern corresponding to the attribute data indicating the attribute of the living body, the environment data acquired by the environment data acquiring unit, or the activity data acquired by the activity data acquiring unit; and
a pattern generating section configured to, upon determination of no first perspiration pattern corresponding to the attribute data, the environment data, or the activity data at the pattern determining section, use a plurality of first perspiration patterns correlated with at least any one of a set of attribute values close to a value indicated by the attribute data, a set of environment values close to a value indicated by the environment data, or a set of activity values close to a value indicated by the activity data to generate a first perspiration pattern used for comparison at the comparing section.

10. The perspiration state estimation device according to claim 1,
wherein the comparing section is configured to perform comparison to identify time corresponding to a value indicated by the local perspiration data in the first perspiration pattern, and
the estimating section is configured to estimate the perspiration state of the site of the living body at a moment when a prescribed time period is elapsed from the time identified as a result of the comparison by the comparing section.

11. The perspiration state estimation device according to claim 1,
wherein the environment data acquiring unit is configured to acquire data indicating at least either of temperature and humidity of the environment, as the environment data.

12. The perspiration state estimation device according to claim 1,
wherein the attribute includes at least any of body-build, age, sex, and cloth information of the living body.

13. A perspiration state estimation method comprising:
a local perspiration data acquiring step of acquiring local perspiration data indicating a perspiration state of a local part of a living body;
an environment data acquiring step of acquiring environment data indicating a state of an environment where the living body is present;
a comparing step of comparing (1) the local perspiration data acquired in the local perspiration data acquiring step with (2) a first perspiration pattern correlated with at least either of attribute data indicating an attribute of the living body and the environment data acquired in the environment data acquiring step, the first perspiration pattern indicating progression of the perspiration state of the local part over time; and
an estimating step of estimating a perspiration state of a site of the living body on a basis of a result of the comparison in the comparing step and a progression relating pattern indicating a second perspiration pattern indicating progression of the perspiration state of the site over time or a progression relating pattern indicating a relationship between the first perspiration pattern and the second perspiration pattern, the site including at least a part other than the local part.

14. A computer-readable recording medium storing a perspiration state estimation program causing a computer to function as the perspiration state estimation device according to claim 1,
wherein the perspiration state estimation program is configured to cause a computer to function as the comparing section and the estimating section.

15. The perspiration state estimation device according to claim 2,
wherein the perspiration state estimation device further includes an identifying section configured to identify at least either of (1) a first perspiration pattern corresponding to the attribute data among a plurality of first perspiration patterns correlated with a plurality of predetermined attribute values indicating the attribute and (2) a first perspiration pattern corresponding to the environment data among a plurality of first perspiration patterns correlated with a plurality of predetermined environment values indicating a prescribed environment state, and
the comparing section is configured to perform the comparison using the first perspiration pattern identified by the identifying section.

16. The perspiration state estimation device according to claim 2,
wherein the local perspiration data acquiring unit is configured to acquire the local perspiration data at a plurality of times, and
the comparing section is configured to perform comparison using plural pieces of the local perspiration data acquired by the local perspiration data acquiring unit.

17. The perspiration state estimation device according to claim 3,
wherein the local perspiration data acquiring unit is configured to acquire the local perspiration data at a plurality of times, and
the comparing section is configured to perform comparison using plural pieces of the local perspiration data acquired by the local perspiration data acquiring unit.

18. The perspiration state estimation device according to claim 15,
wherein the local perspiration data acquiring unit is configured to acquire the local perspiration data at a plurality of times, and
the comparing section is configured to perform comparison using plural pieces of the local perspiration data acquired by the local perspiration data acquiring unit.
